(19) United States
(12) Patent Application Publication
(10) Pub. No.: US 2024/0352508 A1
(43) Pub. Date: Oct. 24, 2024

(54) METHODS, SYSTEMS AND COMPOSITIONS FOR DETECTION OF MULTIPLE ANALYTES

(71) Applicant: ChromaCode, Inc., Carlsbad, CA (US)

(72) Inventors: Christopher MACDONALD, San Diego, CA (US); Jerrod SCHWARTZ, Carlsbad, CA (US)

(21) Appl. No.: 18/681,381

(22) PCT Filed: Aug. 4, 2022
(86) PCT No.: PCT/US2022/039447

§ 371 Date: Feb. 5, 2024

Related U.S. Application Data
(60) Provisional application No. 63/230,569, filed on Aug. 6, 2021.

(51) Int. Cl. C12Q 1/6827; C12Q 1/6858

(52) U.S. Cl. CPC C12Q 1/6827; C12Q 1/6858

(57) ABSTRACT

Methods, systems, compositions and kits for detection of multiple analytes are disclosed. The methods, systems, compositions and kits may comprise an oligonucleotide that may bind to more than one analyte. The oligonucleotide may allow for the generation of different signal depending on the analyte that the oligonucleotide is hybridized to. The signals generated from may be used to identify the presence of an analyte. The signals generated from may be used may be used to simultaneously identify the presence of multiple analytes in a sample.

### CROSS REFERENCE

This application claims the benefit of U.S. Provisional Application No. 63/230,569, filed Aug. 6, 2021, which is incorporated by reference herein in its entirety.

### BACKGROUND OF THE INVENTION

Detection of nucleic acid sequences are used for a wide variety of purposes. Detection of a particular nucleic acid sequence in a gene of a subject may indicate that the subject may have a particular disorder or be more prone to having a particular disorder. Detection of nucleic acid sequences may be used to detect an infection by detecting a gene or nucleic acid sequence of a pathogen. PCR may be used to amplify nucleic acids for analysis.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY

Disclosed herein, in some aspects, are methods, systems and compositions for detection and quantification of multiple analytes.

In an aspect, the present disclosure provides a method of determining the presence or absence of a first analyte and a second analyte in a sample or in a collection of samples, the method comprising: (a) providing a sample solution derived from a sample or collection of samples comprising, or potentially comprising, a first analyte and a second analyte; (b) adding an oligonucleotide configured to hybridize to said first analyte and said second analyte; (c) subjecting said sample solution to an extension reaction, wherein said extension reaction generates a first signal corresponding to said first analyte and/or a second signal corresponding to said second analyte; (d) detecting the presence or absence of said first signal and/or said second signal; and (e) based on said detecting, determining the presence or absence of said first analytes and/or said second analyte. In some embodiments, the determining further comprises comparing said first signal and/or said second signal to a reference signal or a model of expected signals, and determining if said first signal and/or said second signal corresponds to said reference signal or model of expected signals. In some embodiments, the extension reaction comprises (i) hybridizing said oligonucleotide to said first analyte and/or said second analyte and (ii) extending said oligonucleotide. In some embodiments, the first signal and/or said second signal is generated by the extension of said oligonucleotide. In some embodiments, the method further comprises amplifying said first analyte or said second analyte under amplification conditions. In some embodiments, the first signal corresponds with the amplification of said first analyte. In some embodiments, the second signal corresponds with the amplification of said second analyte. In some embodiments, the amplifying comprises a polymerase chain reaction (PCR). In some embodiments, the oligonucleotide acts as a primer in said PCR. In some embodiments, an intensity of a first signal corresponds to the efficiency of (i) hybridizing said oligonucleotide to said first analytes, and (ii) extension of said oligonucleotide. In some embodiments, the method further comprises adding a plurality of probes to said sample solution. In some embodiments, the plurality of probes generates said first signal or said second signal during said extension reaction. In some embodiments, a probe of plurality of probes comprises a detectable label. In some embodiments, the probe of plurality of probes does not comprise a detectable label. In some embodiments, the detectable label is a fluorophore. amplification conditions comprise one or more of thermocycling conditions, a salt concentration, a primer concentration, a deoxynucleotide triphosphate (dNTP) concentration, presence of a polymerase, and a template concentration. In some embodiments, wherein under said amplification conditions, the amplifying of said first analyte occurs at a higher rate and/or efficiency compared to said amplifying of said second analyte. In some embodiments, the first signal, if generated has a higher intensity than said second signal, if generated. In some embodiments, the determining said presence or absence of said first or second analyte comprises processing the intensity of said first signal or second signal. In some embodiments, the collection of samples is derived from a subject. In some embodiments, the collection of samples is derived from a plurality of subjects. In some embodiments, the subject is an biological organism or virus. In some embodiments, the biological organism is a human, animal, plant, fungus, bacteria or archaea. In some embodiments, (e) further comprises determining the presence or absence of said first analyte or second analyte in said plurality of subjects. In some embodiments, prior to (a), said collection of samples is pooled to generate said sample solution. In some embodiments, the second analyte is a variant of said first analyte. In some embodiments, the first analyte is a wild-type sequence and said second analyte is a mutant sequence. In some embodiments, the first and second analyte differ from one another by at least one nucleotide. In some embodiments, the first and second analyte differ from one another by at most one nucleotide. In some embodiments, the first and second analyte differ from one another by at a single nucleotide polymorphism. In some embodiments, the first and second analyte differ from one another by a plurality of single nucleotide polymorphisms. In some embodiments, the first and second analyte differ from one another by a deletion, insertion, transversion, duplication, or a combination thereof. In some embodiments, the deletion is at least 1, 2, 3, 4, 5, 6, 7, 8, 9, 10, 15, 20, 25, or more nucleotides in length. In some embodiments, the insertion is at least 1, 2, 3, 4, 5, 6, 7, 8, 9, 10, 15, 20, 25, or more nucleotides in length. In some embodiments, the transversion is at least 1, 2, 3, 4, 5, 6, 7, 8, 9, 10, 15, 20, 25, or more nucleotides in length. In some embodiments, the duplication is at least 1, 2, 3, 4, 5, 6, 7, 8, 9, 10, 15, 20, 25, or more nucleotides in length. In some embodiments, the first and second analytes comprise a sequence identity to one another of at least 80%, at least 90%, at least 95%, or at least 99%. In some embodiments, the method further comprises, prior to (c), partitioning the sample into a plurality of partitions. In some embodiments, a partition of said plurality of partition comprises said first analyte or said second analyte. In some embodiments, each partition of said plurality of partition comprises on average one copy of said first analyte and/or said second analyte. In some embodiments, each partition of said plurality of partition comprises one copy or less of said first analyte and/or said second analyte. In some embodiments, the partitioning comprises co-partitioning said oligonucleotide and said first analyte or said second analyte in a partition. In some embodiments, the first signal and/or said second signal comprises a kinetic signature for said partition. In some embodiments, the method further comprises comparing said kinetic signature for said partition with another kinetic signature of a partition of said subset of said partition. In some embodiments, the comparing comprises determining the presence of said first analyte and said second analyte if said kinetic signature and said another kinetic signature differ. In some embodiments, the method further comprises quantifying the amount first analyte or second analytes. In some embodiments, the method further comprises quantifying a relative amount of first analyte to second analytes. In some embodiments, the first analyte or said second analyte corresponds to a disease condition in a source of said sample or collection of samples. In some embodiments, the sample or collection of samples is derived from wastewater. In some embodiments, the sample or collection of samples is derived from soil. In some embodiments, the first analyte or said second analyte corresponds to a genetic variant. In some embodiments, the first analyte or said second analyte comprises a human genomic sequence. In some embodiments, the first analyte or said second analyte comprises an animal genomic sequence. In some embodiments, the first analyte or said second analyte comprises a plant genomic sequence. In some embodiments, the first analyte or said second analyte comprises a fungal genomic sequence. In some embodiments, the first analyte or said second analyte comprises an archaeal genomic sequence. In some embodiments, the first analyte or said second analyte comprises a pathogen associated sequence. In some embodiments, the pathogen-associated sequence comprises a viral sequence. In some embodiments, the pathogen-associated sequence comprises a bacterial sequence. In some embodiments, the first analyte is derived from a first pathogen and the second analyte is derived from a second pathogen. In some embodiments, the first pathogen and said second pathogen are a same pathogen. In some embodiments, the first pathogen and said second pathogen are a different pathogen. In some embodiments, the first pathogen or said second pathogen is a coronavirus. In some embodiments, the coronavirus is SARS-COV-2. In some embodiments, the first pathogen or said second pathogen is an influenza virus. In some embodiments, the first pathogen or said second pathogen is a respiratory syncytial virus. In some embodiments, the first pathogen or said second pathogen is a pathogen associated with respiratory infections. In some embodiments, the sample is derived from a plurality of cells. In some embodiments, the plurality of cells are derived from a tumor. In some embodiments, the sample is a plasma sample or blood sample. In some embodiments, the sample comprises maternal nucleic acids and fetal nucleic acids. In some embodiments, the first signal or second signal is an endpoint signal. In some embodiments, the method further comprising, generating a kinetic signature from said first signal or said second signal. In some embodiments, the kinetic signature comprises measuring a signal and a time point at a series of temperatures. In some embodiments, the kinetic signature is represented as a curve based on endpoint signal over a series of temperatures. In some embodiments, the method further comprises generating a kinetic signature from said first signal and said second signal. In some embodiments, the oligonucleotide comprises a hairpin. In some embodiments, the oligonucleotide forms a hairpin upon hybridizing to said first analyte. In some embodiments, the oligonucleotide does not form a hairpin upon hybridizing to said second analyte. In some embodiments, the oligonucleotides is configured to form a hairpin in the presence of the first analyte, and not form a hairpin in the presence of said second analyte.

In another aspect, the present disclosure provides an oligonucleotide for determining the presence or absence of a first analyte and a second analyte, wherein said oligonucleotide is configured to hybridize to said first analyte and said second analyte, wherein said oligonucleotide generates a first signal corresponding to said first analyte and a second signal corresponding to said second signal. In some embodiments, the oligonucleotide is a primer. In some embodiments, extension of said oligonucleotides generates said first signal and said second signal. In some embodiments, the oligonucleotide is a probe. In some embodiments, the oligonucleotide comprises a detectable label. In some embodiments, a hybridization efficiency of said oligonucleotide to said first analytes is different than a hybridization efficiency said oligonucleotide to said second analyte.

In another aspect, the present disclosure provides a kit for determining the presence or absence of a first analyte and a second analyte, wherein the kit comprises: (a) a primer oligonucleotide configured to hybridize to said first analyte and said second analyte, wherein said oligonucleotide generates a first signal corresponding to said first analyte and a second signal corresponding to said second analyte and (b) a probe oligonucleotide configured to bind to said first analyte or said second analyte.

In another aspect, the present disclosure provides a kit for determining the presence or absence of a first analyte and a second analyte, wherein the kit comprises: (a) a probe oligonucleotide configured to hybridize to said first analyte and said second analyte, wherein said probe oligonucleotide generates a first signal corresponding to said first analyte and a second signal corresponding to said second analyte and (b) a set of primer oligonucleotide configured to bind to said first analyte or said second analyte.

In another aspect, the present disclosure provides a method of determining the presence or absence of a first analyte and a second analyte in a sample or in a collection of samples, the method comprising: (a) providing a sample solution comprising, or potentially comprising at a first analyte and a second analyte; (b) adding an oligonucleotide that is configured to hybridize to said first analyte and said second analyte; (c) subjecting said sample to an extension reaction, wherein said extension reaction generates: (i) a first signal if said first analyte is present or (ii) a second signal if said second analyte is present and the first analyte is not present; (d) detecting the first or second signal; and (e) based on said detecting of said first signal or said second signal, determining said presence or absence of said first and second analyte

In another aspect, the present disclosure provides a method of determining the presence or absence of a target nucleotide sequence, wherein the method comprises: (a) providing a sample solution comprising, or potentially comprising, an analyte sequence comprising said target nucleotide sequence and a second analyte comprising a reference sequence; (b) adding an oligonucleotide to a sample solution, wherein said oligonucleotide is configured to hybridize to a reference sequence and said target nucleotide sequence; (c) subjecting said sample to an extension reaction, wherein said extension reaction generates a signal corresponding to said target nucleotide sequence and a reference signal corresponding to said reference sequence; (d) detecting said signal and comparing said signal to a reference or model signal corresponding to said reference sequence; (e) based on said comparing, determining the presence or absence of a target sequence.

In another aspect, the present disclosure provides a method of determining the presence or absence of a first analyte and a second analyte in a sample, the method comprising: (a) providing said sample; (b) adding to said sample an oligonucleotide configured to hybridize to said first analyte and said second analyte; (c) subjecting said sample to a first extension reaction at a first reaction condition, wherein said extension reaction generates a first plurality of signals corresponding to the extension products generated by said first extension reaction; (d) measuring, said first plurality of signals, if present; (e) subjecting said sample to a second extension reaction at a second reaction condition, wherein said extension reaction generates a second plurality of signals corresponding to the extension products generated; (f) measuring, said second plurality of signal, if present; and (g) based on the measuring of said first plurality of signals and said second plurality of signals, determining said presence or absence of said first and second analytes. In some embodiments, the first reaction condition comprises a first annealing condition and said second reaction condition comprises second annealing condition. In some embodiments, the first annealing condition is more stringent than said the second annealing condition. In some embodiments, the first annealing condition comprises an annealing temperature that is greater than an annealing temperature of the second annealing condition. In some embodiments, the first annealing condition comprises an annealing time that is greater than an annealing time of the second annealing condition. In some embodiments, the first reaction condition comprises a first extension condition and said second reaction condition comprises second extension condition. In some embodiments, the first extension condition or second extension condition is an extension time or temperature. In some embodiments, the first annealing condition comprises an annealing temperature that is greater than an annealing temperature of the second annealing condition. In some embodiments, wherein in (c), said hybridization of said oligonucleotide to said first analyte is more favorable than hybridization of said oligonucleotide to said second analyte. In some embodiments, wherein in (e), said oligonucleotide hybridizes to said first analyte and said second analyte.

### BRIEF DESCRIPTION OF THE DRAWINGS

An understanding of the features and advantages of the present invention will be obtained by reference to the following detailed description that sets forth illustrative embodiments, in which the principles of the invention are utilized, and the accompanying drawings of which:

FIG. 1. shows PCR curves for different samples comprising an analyte.

### DETAILED DESCRIPTION OF THE INVENTION

The following description provides specific details for a comprehensive understanding of, and enabling description for, various embodiments of the technology. It is intended that the terminology used be interpreted in its broadest reasonable manner, even where it is being used in conjunction with a detailed description of certain embodiments.

Before describing the present teachings in detail, it is to be understood that the disclosure is not limited to specific compositions or process steps, and as such, may vary. As used herein, the singular forms “a,” “an,” and “the” are intended to include the plural forms as well, unless the context clearly indicates otherwise. Furthermore, to the extent that the terms “including,” “includes,” “having,” “has,” “with,” “such as,” or variants thereof, are used in either the specification and/or the claims, such terms are not limiting and are intended to be inclusive in a manner similar to the term “comprising.” Unless specifically noted, embodiments in the specification that recite “comprising” various components are also contemplated as “consisting of” or “consisting essentially of” the recited components.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated

The term “subject,” as used herein, generally refers to an animal, such as a mammal (e.g., human) or avian (e.g., bird), or other organism, such as a plant. For example, the subject can be a vertebrate, a mammal, a rodent (e.g., a mouse), a primate, a simian or a human. A subject can be a healthy or asymptomatic individual, an individual that has or is suspected of having a disease (e.g., cancer) or a pre-disposition to the disease, and/or an individual that is in need of therapy or suspected of needing therapy. A subject can be a patient.

The term “channel,” “color channel,” or “optical channel”, as used herein, generally refers to a range of wavelengths. The channel may be set or determined based on particular filters which remove or filter out particular wavelengths. The terms “channel,” “color channel,” and “optical channel” can be used interchangeably.

Polymerase Chain Reaction (PCR) is a method of exponential amplification of specific nucleic acid target in a reaction mix with a nucleic acid polymerase and primers. Primers are short single stranded oligonucleotides which are complementary to the 3′ sequences of the positive and negative strand of the target sequence. The reaction mix is cycled in repeated heating and cooling steps. The heating cycle denatures or splits a double stranded nucleic acid target into single stranded templates. In the cooling cycle, the primers bind to complementary sequence on the template. After the template is primed the nucleic acid polymerase creates a copy of the original template. Repeated cycling exponentially amplifies the target 2-fold with each cycle leading to approximately a billion-fold increase of the target sequence in 30 cycles (Saiki et al 1988).

Real-Time PCR (qPCR) is a process of monitoring a PCR reaction by recording the fluorescence generated either by an intercalating dye such as SYBR Green or a target-specific reporter probe at each cycle. This is generally performed on a Real-Time PCR instrument that executes thermal cycling of the sample to complete the PCR cycles and at a specified point in each cycle measures the fluorescence of the sample in each channel through a series of excitation/emission filter sets.

Digital PCR (dPCR) is a process of partitioning a sample containing one or more targets into a plurality of partitions (e.g., wells, droplets, arrays, etc.), performing a PCR reaction in each partition, and recording the luminescence (e.g., fluorescence) generated by, for example, a target-specific re-porter probe. The use of labeled oligonucleotide probes enables specific detection. dPCR may be used in a variety of nucleic acid detection methods. Digital PCR is generally performed on a digital PCR instrument that measures the fluorescence from each partition in an optical channel through one or more excitation/emission filter sets. Frequently, the target-specific oligonucleotide probe is a short oligonucleotide complementary to one strand of the amplified target. The probe may lack a 3′ hydroxyl and therefore is not extendable by the DNA polymerase. TaqMan (ThermoFisher Scientific) chemistry is a common reporter probe method used for multiplex Real-Time PCR (Holland et al. 1991). The TaqMan oligonucleotide probe is covalently modified with a fluorophore and a quenching tag (i.e., quencher). In this configuration the fluorescence generated by the fluorophore is quenched and is not detected by the real time PCR instrument. When the target of interest is present, the probe oligonucleotide base pairs with the amplified target. While bound, it is digested by the 5′ to 3′ exonuclease activity of the Taq polymerase thereby physically separating the fluorophore from the quencher and liberating signal for detection by the real time PCR instrument.

Primers, or “amplification oligomers,” used herein interchangeably, refer to an oligonucleotide or nucleic acid configured to bind to another nucleic acid and facilitate one or more reactions, for example, transcription, nucleic acid synthesis, and nucleic acid amplification. A primer can be double-stranded. A primer can be single-stranded. A primer can be a forward primer or a reverse primer. A forward primer and a reverse primer can be those which bind to opposite strands of a double-stranded nucleic acid. For example, a forward primer can bind to a region of a first strand (e.g., Watson strand) derived from a nucleic acid, and a reverse primer can bind to a region of a second strand (e.g., Crick strand) derived from the nucleic acid. A forward primer may bind to a region closer to the start site of a gene relative to a reverse primer or may bind closer to the end site of a gene relative to a reverse primer. A forward primer may bind to the coding strand of a nucleic acid or may bind to the non-coding strand of a nucleic acid. A reverse primer may bind to the coding strand of a nucleic acid or may bind to the non-coding strand of a nucleic acid.

Provided herein are methods for detection of multiple nucleic acids sequences or analytes in a sample. The methods may comprise the use of an oligonucleotide configured or otherwise able to hybridize to more than one analyte. The oligonucleotide that may hybridize to more than one analyte may allow the generation of a signal corresponding to the hybridization to a first analyte and a second signal generated based on the hybridization to a second analyte. The detection of a signal may indicate the presence of a first analyte, a second analyte, or both. The detection of nucleic acids sequences may indicate a disease state, disorder, or the presence of a pathogen. The use of an oligonucleotide able to hybridize to more than one analyte may allow an oligonucleotide to be used to analyze multiple different analytes and may allow for the detection of multiple variants in a single sample.

Provided herein are oligonucleotides that are able to hybridize to more than one analyte and allow the generation of a distinct signal depending on which analytes has been hybridized. The hybridization of the oligonucleotide to a first analyte may allow a first signal to be generated, whereas the hybridization of the oligonucleotide to a second analyte may generate a second signal. The first and second signal may be differentiable such that detection of a signal would allow for the determination of the presence of a given analyte. Based on detecting of the signal, it may be possible to determine which analyte has been hybridized to, and thereby allow detection of a specific analyte. The oligonucleotides may comprise a hairpin. The oligonucleotide may form a hairpin upon hybridization with an analyte. The oligonucleotide may selectively form a hairpin upon hybridization with an analyte but not form a hairpin with another analyte.

In various aspects disclosed herein, the results of the assay may be used to determine that a nucleic assay sequence or infectious pathogen is present in a subject or sample. The assay may be used to generate a genotype or determine the presence of an infectious pathogen in a subject or diagnose the presence of a disorder or disease. The assay may be able to detect the presence of a microbe in an environmental sample. The assay may be able to determine the presence of a nucleic acid sequence in a plurality of subjects.

In various aspects disclosed herein, the results of the assay may be used to generate a risk score or probability of risk. Using the results of assay, for example, the genotype of an individual or the presence of a genetic marker may indicate that a subject is at a higher risk for infection, or a higher risk of complications or mortality due to an infection. The markers may be related to immune system response or a related to proteins involved in or targeted by viruses for entry or other pathways for infection. The results of the assay may indicate that a pathogen is absent or present. The results may indicate that a particular strain or serotype is present. The results may be combined with other data relating to the subject medical history.

In various aspects described herein a sample is a collected from a subject or a plurality of subjects. The biological sample may be collected using a sample collection tube or vessel. The biological sample may be collected using a sample collection tool. The sample collection tool may comprise swab. The sample may be collected by the subject without the help of another individual. The sample may be collected in a subject's home away from a medical facility. The sample may be collected based on a set of instructions provided in a kit to the subject.

In various aspects described herein, the results of the assay may be outputted as a report. The report may be outputted to a remote computer database. The report may be outputted such that it is accessible to the subject. The report may be outputted such that it is accessible to a medical provider or to an institution. The report may be accessible via a smart phone. The report may be accessible via an application. The report may be used in the conjunction with a subject medical record. The report may comprise medical recommendations.

Methods as described herein may be used to quantify 1, 2, 3, 4, 5, 6, 7, 8, 9, 10, or more analytes in a sample volume. First, a mixture may be provided comprising a plurality of nucleic acid molecules and a plurality of oligonucleotide probes. The plurality of nucleic acid molecules may be derived from, and/or may correspond with, the nucleic acid target in the sample. The plurality of oligonucleotide probes may each correspond to a different region of the nucleic acid target. The oligonucleotide probes be able to hybridize to more than one nucleic acid target. The mixture may further comprise other reagents (e.g., amplification reagents) including, for example, oligonucleotide primers, dNTPs, a nucleic acid enzyme (e.g., a polymerase), and salts (e.g., Ca2+, Mg2+, etc.). A oligonucleotide primer of the oligonucleotide primers may be able to hybridize to more than one nucleic acid target. The mixture may be partitioned into a plurality of partitions. Next, the mixture may be used in a quantitative Polymerase Chain Reaction (qPCR), whereby a plurality of signals may be generated. The plurality of signals may be detectable in multiple color channels. Based on the detecting, the nucleic acid target in the sample may be quantified. A signal of the plurality of the signal may be detectable in only one color channel. For example, a first signal of the plurality of signals is detected in multiple color channels, and a second signal is detectable in only one color channel, and the analytes correlated to the first and second signals may be quantified. In another example, a first signal of the plurality of the signals is detected in a first two color channels and a second signal of the plurality of signals is detected in a second two color channels, and at least one of the channels in the first two color channels and the second two color channels is the same or substantially the same color channel. In another example, a first signal may be generated that corresponds to a first analyte and a second signal may be generated that corresponds to a second analyte. The different signal may then be analyzed to determine the presence of the analytes, as described elsewhere herein. In the case of a partitioned sample, the plurality of signals may be detected in multiple partitions of the plurality of partitions. A signal may be detected or measured at least 1, 2, 3, 4, 5, 6, 7, 8, 9, 10, 11, 12, 13, 14, 15, 20, 30, 40, 50, or more channels. A signal may be detected or measured in no more than 1, 2, 3, 4, 5, 6, 7, 8, 9, 10, 11, 12, 13, 14, 15, 20, 30, 40, 50, or less channels.

In various examples, an analyte to be detected is encoded as or correlated to a value of a signal (e.g., a signal intensity). The values may be assigned so that the results of the assay unambiguously indicate the presence or absence of the analytes being assayed. For example, a first analyte may be correlated or encoded as an intensity of 1i (or i), wherein a second analyte may be correlated or encoded as an intensity of 2i. The detection of a signal intensity of 1i may indicate that the presence of the first analyte whereas the detection of a signal intensity of 2i may indicate the presence of the second analyte. In other examples, each analyte to be detected is encoded or correlated as a value in each of at least two components of a signal (e.g., intensity and wavelength). The at least two components of a signal may be orthogonal. For example, a first analyte may be encoded or correlated to an intensity of 1i and a wavelength of n, whereas a second analyte may be encoded or correlated to an intensity of 1i and a wavelength of m. In another example, a first analyte may be encoded or correlated to an intensity of 1i and a wavelength of n, whereas a second analyte may be encoded or correlated to an intensity of 2i and a wavelength of n.

The plurality of signals may be generated by one or more of the plurality of probes from the mixture. The plurality of signals may be generated by a probe able to bind to multiple analytes. The plurality of signals may be generated by nucleic acid amplification (e.g., PCR) of the plurality of nucleic acid molecules. Nucleic acid amplification may degrade the plurality of oligonucleotide probes (e.g., by activity of a nucleic acid enzyme), thereby generating the plurality of signals. A plurality of signals may be a plurality of fluorescent signals, a plurality of chemiluminescent signals, or a combination thereof.

A cumulative signal measurement may be generated comprising one or more signals generated from a plurality of hybridization probes. A hybridization probe may correspond to a polynucleotide analyte (e.g., may bind to a region of a polynucleotide analyte). One or more additional cumulative signal measurements may be generated, each comprising one or more additional signals generated from the one or more additional pluralities of hybridization probes. A cumulative signal measurement may comprise one or more signals generated from one or more probes provided to a sample solution. A cumulative signal measurement may be a signal intensity level which corresponds to the sum of signals generated from multiple oligonucleotide probes. For example, two probes may each bind to a nucleic acid molecule, where each probe generates a signal of a given wavelength at 1× intensity. Measurement of these signals would generate a cumulative signal measurement corresponding to the sum of both signal intensities, namely a 2× signal intensity.

Signal intensities correlated to analyte may additionally be modulated by the addition of competitive nucleic acids that may bind to the analyte. A competitive nucleic acid may compete with oligonucleotide probe for a given analyte to reduce the signal intensity generated for an analyte. A competitive nucleic acid may lack a fluorophore or other signal generating tag and may compete for binding of an analyte with a probe that comprises a fluorophore. With the binding of the competitive nucleic acid as opposed to the binding of a probe, the fluorescent intensity associated with the presence of the analyte may be decreased.

In multiple aspects as described herein, signals and data relating to the detection of the signals are subjected to processing in order for the signals and data to be used for subsequent steps or downstream methods. The processing may use mathematical algorithms to analyze or process the signal data. In some case, the processing may use data obtained from the instrument or detector. The processing may use data obtained from multiple channels, or a single channel. In some cases, the processing may use data from channels that are not expected to correlate with a signal from a given probe or fluorophore. For example, the data may include data obtained from a reference channel in which a background signal is obtained. The processing may use data obtained from all available channels of a given detection device.

The mathematical algorithms used for data processing may include expectation maximization, nearest neighbor analysis, basic model parameterization, Bayesian estimation, or combinations thereof. The mathematical algorithm may use a process parameter. Examples or process parameters include parameters for threshold cycles, amplitudes, or slopes.

The processing of data may comprise plotting the data. Processing the data may use plotting functions to analyze individual or multiple points such to calculate a correlation or to better visualize data. The data may be plotted as a curve. The data may be represented as a kinetic signature, wherein the signal amplitude may plotted be against a metric of time (such as cycles or seconds) or a metric that can be mathematically transformed into a metric of time (such as a frequency). The data may be fit to a variety of functions in order to derive parameters from the data. For example, a plotted data may be fit to a linear function such that a slope parameter can be derived from the data.

Kinetic signatures of the signals and data may be generated. Kinetic signatures may be generated by plotting an intensity of the signal against a time. For example, a kinetic signature mat be generated by plotting the signal intensity generated by a probe or intercalating dye at a given cycle. Kinetic signatures may be generated by plotting an endpoint signal at multiple temperatures. The kinetic signatures may show the amplification kinetics of a reaction, which may vary between reaction of different analytes. Reactions using the oligonucleotide that may hybridize to multiple analytes may have a kinetic signature corresponding to a first analyte and another kinetic signature corresponding to a second analyte. Kinetic signatures may be generated for analytes in a sample. Kinetic signatures from in a sample may be compared with kinetic signals from reference kinetic signatures and may indicate the presence an analytes in the sample. For example, in a first a first analyte may be present and generate a first signal and/or a second analyte may be present and generate a second signal. The signals may be used to generate a kinetic signature or curve shape. By observing the presence of the first signal and/or the presence of a second signal, the presence of either analyte or both analytes may be identified. Kinetic signatures from a first analyte may be compared to kinetic signatures of a second analyte. The kinetic signatures may comprise different curve shapes such that a first kinetic signature may be distinguished from a second kinetic signature. The kinetic signatures may comprise different endpoints such that a first kinetic signature may be distinguished from a second kinetic signature. The kinetic signatures may comprise a same or similar endpoint with different curve shapes such that a first kinetic signature may be distinguished from a second kinetic signature.

Processing of the data may also comprise identifying a data point as belonging to a data set. In some cases, multiple analytes are analyzed simultaneously, wherein the signal generated from analytes may comprise overlapping signals from different analytes. Processing the data may alleviate the overlapping signals or may correlate the data points to different data set in which the signal is detected via another method or alternative channel or detector.

In various aspects, reference signals or models of expected signals may be used for comparing with signals or data sets. By comparing a signal or data set to a reference signal or model of expected signals, an analyte may be identified as present in the sample. For example a reference signal may be generated by running a reaction with a known analyte. The signal may be compared to the reference signal and based on if the signal and reference signal match, a determination regarding the presence of the known analyte may be made. Reference signals may be generated using reference conditions. A reference condition may comprise a known concentration of a reagent or analytes. Reference conditions may comprise a known reaction condition such as the temperature or pH of a solution. For example, the reference condition may comprise a concentration, amount, or quantity of a reference nucleic acid. The reference condition with the known parameter may be used to extrapolate, interpolate or otherwise calculate a concentration, quantity, or amount of another nucleic acid in a separate sample. Reference conditions may comprise signals which may be detected or processed or as described elsewhere herein for any other signal. For example, data from the reference condition may be used to generate reference data which in turn may be parameterized by mathematical algorithms to generate reference quantification parameters. The generation of reference quantification parameters can be used to directly compare to generated quantification parameters of a data set or can be used to calculate a quantification parameter based on for example, parameterization, fitting, extrapolation, interpolation, or estimation of the data set or a parameter of the data set.

References conditions may be specific to a type of reaction. Reference conditions may comprise conditions for an amplification reaction. Examples of amplification reactions are described elsewhere herein. Reference conditions may, for example, comprise a concentration of a polymerase or a type of polymerase. For example, reference conditions may comprise a) a primer concentration, b) a polymerase concentration, c) polymerase type, d) a reference nucleic acid concentration, e) a number of thermocycles, f) a rate of thermocycling, g) a thermocycle time length, h) a probe sequence; i) a primer sequence, or combinations thereof.

In some cases, the sample further comprises an additional plurality of nucleic acid molecules and an additional plurality of oligonucleotide probes. The additional plurality of nucleic acid molecules may be derived from and/or correspond with an additional nucleic acid target. The additional plurality of oligonucleotide probes may each correspond to a different region of the additional nucleic acid target.

In various aspects, nucleic acid molecules may be quantified. The quantification may be an absolute quantification. For example, the molarity of a starting amount of a nucleic acid may be determined. This may be determined using a reference condition or amount with a known molarity of nucleic acid. The quantification may be a relative quantification. For example, a second nucleic acid may be determined to have a larger starting amount than a first nucleic acid.

A sample may be a biological sample. A sample may be derived from a biological sample. A biological sample may be, for example, blood, plasma, serum, urine, saliva, mucosal excretions, sputum, stool or tears. A biological sample may be a fluid sample. A fluid sample may be blood or plasma. A biological sample may comprise cell-free nucleic acid (e.g., cell-free RNA, cell-free DNA, etc.). A nucleic acid target may be a nucleic acid from a pathogen (e.g., virus, bacteria, etc.). A nucleic acid target may be a nucleic acid suspected of comprising one or more mutations.

In various aspects, partitioning is performed and partitions are generated. Reactions described elsewhere in the specification may be performed in the partitions. For example, a sample containing at least one nucleic acid target sequence, at least one amplification oligonucleotide, at least one detection oligonucleotide, dNTPs, a thermostable DNA polymerase, and other PCR reagents may be partitioned into a plurality of partitions. A partition may be a droplet (e.g., a droplet in an emulsion). A partition may be a microdroplet. A partition may be a well. A partition may be a microwell. Partitioning may be performed using a microfluidic device. In some cases, partitioning is performed using a droplet generator. Partitioning may comprise dividing a sample or mixture into water-in-oil droplets. A droplet may comprise one or more nucleic acids. A droplet may comprise a single nucleic acid. A droplet may comprise two or more nucleic acids. A droplet may comprise no nucleic acids. Each droplet of a plurality of droplets may generate a signal. A plurality of signals may comprise the signal(s) generated from each of a plurality of droplets comprising a subset of a sample.

Generally, each partition may receive a single template of the nucleic acid target sequence. However, statistically, some partitions may receive more than one copy of a nucleic acid target template, while other partitions may not receive any target template. The detection oligonucleotide or amplification oligonucleotide may be configured to bind to multiple analytes. The partitions may each comprise at most one analyte. In each partition, reaction may be performed to generate a signal and may indicate the analyte in a given partition. Kinetic signatures may be generated for each partition. Kinetic signatures from a given partition may be compared with kinetic signals from other partitions and may indicate the presence of more than one analyte in the sample. For example, in a first partition a first analyte may be present and generate a first signal. In a second partition a second analyte may be present and generate a second signal. The signals may be used to generate a kinetic signature or curve shape. By observing the presence of the first signal in a first partition and the presence of a second signal in a second partition, the presence of both analytes may be identified. The methods and reactions described elsewhere may be used in conjunction with partitions, such that an analyte in a partition may be identified, quantified or characterized as described elsewhere herein.

In some embodiments, the present disclosure provides a multiplexed assay for simultaneous amplification, detection, and or/quantification of one or more analyte in a sample. The one or more analytes may comprise different sequences from one another. As described elsewhere herein an oligonucleotide may be able hybridize to a first analyte and a second analyte that are different. For example, a first analyte may be a variant of second analyte. A first analyte may be a wild type sequence and a second analyte may be a mutant sequence. A first analyte and a second analyte may differ from one another by at least one nucleotide. A first analyte and a second analyte may differ from one another by at most one nucleotide. A first and second analyte may differ from one another by a single nucleotide polymorphism. A first and second analyte may differ from one another by a plurality of single nucleotide polymorphism. A first and second analyte may differ from one another by a deletion, insertion, transversion, duplication, or a combination thereof. A deletion may comprise at least 1, 2, 3, 4, 5, 6, 7, 8, 9, 10, 15, 20, 25, or more nucleotides in length. An insertion may comprise at least 1, 2, 3, 4, 5, 6, 7, 8, 9, 10, 15, 20, 25, or more nucleotides in length. A transversion may comprise at least 1, 2, 3, 4, 5, 6, 7, 8, 9, 10, 15, 20, 25, or more nucleotides in length. A duplication may comprise at least 1, 2, 3, 4, 5, 6, 7, 8, 9, 10, 15, 20, 25, or more nucleotides in length. A first and second analytes may comprise a sequence identity to one another of at least 80%, at least 90%, at least 95%, or at least 99%.

In some cases, assays may be run using the reagents in the chemical composition. Assay may use a reagent to perform a reaction. The reaction may comprise a hybridization reaction. For example, the reagent may comprise a nucleic acid and hybridize with another nucleic acid. The reaction may comprise an extension reaction. For example, the reaction may comprise extending a nucleic molecule by the addition of a nucleotide. The reaction may comprise a polymerase chain reaction.

Methods as described herein may be performed without the use of immobilization, separation, mass spectrometry, or melting curve analysis. For example, the sample reagents and analytes may all be in solution. The analytes may be analyzed without needing to purify or physically separate the analytes from one another. Identification of the analytes may be performed without obtaining a mass of the analytes via mass spectrometry or any similar technique. Additionally, the methods may be used without observing a melting reaction and plotting the signal against a temperature. For example, an analyte may be identified without subjecting the analyte to temperature gradient in order to analyze a specific temperature in which an analyte goes through a physical or chemical change. The methods as described herein may be corroborated via techniques using immobilization, separation, mass spectrometry, or melting curve analysis. For example, the melting curve may be used to verify a number of different amplicons or detecting a presence of an amplicon.

Any number of nucleic acid targets may be detected using assays of the present disclosure. In some cases, an assay may unambiguously detect at least 3, 4, 5, 6, 7, 8, 9, 10, 11, 12, 13, 14, 15, 20, 30, 40, 50 nucleic acid targets, or more. In some cases, an assay may unambiguously detect at most 50, 40, 30, 20, 15, 14, 13, 12, 11, 10, 9, 8, 7, 6, 5, 4, or 3 nucleic acid targets. An assay may comprise any number of reactions, where the results of the reactions together identify a plurality of nucleic acid targets, in any combination of presence or absence. An assay may comprise 2, 3, 4, 5, 6, 7, 8, 9, 10 reactions, or more. Each reaction may be individually incapable of non-degenerately detecting the presence or absence of any combination of nucleic acid targets. However, the results of each reaction together may unambiguously detect the presence or absence of each of the nucleic acid targets.

Reactions may be performed in the same sample solution volume. For example, a first reaction may generate a fluorescent signal in a at least a first color channel, while a second reaction may generate a fluorescent signal in a second color channel, thereby generating two measurements for comparison. Alternatively, reactions may be performed in different sample solution volumes. For example, a first reaction may be performed in a first sample solution volume and generate a fluorescent signal in at least two channels, and a second reaction may be performed in a second sample solution volume and generate a fluorescent signal in the same color channel or a different color channel, thereby generating two measurements for comparison.

An oligonucleotide probe may be labeled with a detectable label. A detectable label may comprise a fluorophore. Fluorescent molecules may be excited at a wavelength at emit light at another wavelength. The fluorescent molecules may be visible to the naked human eye. The fluorescent molecules may visible or identified via spectroscopic methods such to analyze the wavelength of light that are transmitted or absorbed by a solution comprising a fluorescent molecule.

The fluorescent molecules may have a distinct or known signature of excitation or emission wavelength of electromagnetic radiation. The detection of a fluorescent molecule signature may comprise identifying an amplitude or amplitudes of signal at different wavelengths. In some cases, the fluorescent molecule signature may comprise a signal at wavelengths that overlaps with wavelengths that may be generated by reagents in the chemical composition. In some cases, the excitation wavelength of the molecule may comprise a signal that overlaps with wavelengths that may be generated by reagents in the chemical composition. In some cases, the signals of the reaction and the fluorescent molecule may be simultaneously detected. Non-limiting examples of fluorescent molecules that may be used include Alexa Fluor 350, Alexa Fluor 350, Alexa Fluor 405, Alexa Fluor 488, Alexa Fluor 532, Alexa Fluor 546, Alexa Fluor 555, Alexa Fluor 568, Alexa Fluor 594, Alexa Fluor 647, Alexa Fluor 680, Alexa Fluor 750, Cy3, Cy5, Texas Red, Fluorescein (FITC), 6-FAM, 5-FAM, HEX, JOE, TAMRA, ROX, BODIPY FL, Pacific Blue, Pacific Green, Coumarin, Oregon Green, Pacific Orange, Trimethylrhodamine (TRITC), DAPI, APC, Cyan Fluorescent Protein (CFP), Green Fluorescent Protein (GFP), Red Fluorescent Protein (RFP), Phycoerythin (PE), quantum dots (for example, Qdot 525, Qdot 565, Qdot 605, Qdot 705, Qdot 800), or derivatives thereof.

### Amplification

In some aspects, the disclosed methods comprise nucleic acid amplification. Amplification conditions may comprise thermal cycling conditions, including temperature and length in time of each thermal cycle. The use of particular amplification conditions may serve to modify the signal intensity of a signal, thereby enabling a signal (or plurality of signals) to correspond to a unique combination of nucleic acid targets. Amplification may comprise using enzymes such to produce additional copies of a nucleic. The amplification reaction may comprise using oligonucleotide primers as described elsewhere herein. The oligonucleotide primers may use specific sequences to amplify a specific sequence. The oligonucleotide primers may amplify a specific sequence by hybridizing to a sequence upstream and downstream of the primers and result in amplifying the sequence inclusively between the upstream and downstream primer. The oligonucleotide may be able to amplify more than one sequence analyte by hybridizing upstream or downstream of multiple different sequences. The amplification reaction may comprise the use of nucleotide tri-phosphate reagents. The nucleotide tri-phosphate reagents may comprise using deoxyribo-nucleotide tri-phosphate (dNTPs). The nucleotide tri-phosphate reagents may be used as precursors to the amplified nucleic acids. The amplification reaction may comprise using oligonucleotide probes as described elsewhere herein. The amplification reaction may comprise using enzymes. Non-limiting examples of enzymes include thermostable enzymes, DNA polymerases, RNA polymerases, and reverse transcriptases. The amplification reaction may comprise generating nucleic acid molecules of a different nucleotide types. For example, a target nucleic acid may comprise DNA and an RNA molecule may be generated. In another example, an RNA molecule may be subjected to an amplification reaction and a cDNA molecule may be generated.

### Thermal Cycling

Methods of the present disclosure may comprise thermal cycling. Thermal cycling may comprise one or more thermal cycles. Thermally cycling may be performed under reaction conditions appropriate to amplify a template nucleic acid with PCR. Amplification of a template nucleic acid may require binding or annealing of oligonucleotide primer(s) to the template nucleic acid. Appropriate reaction conditions may include appropriate temperature conditions, appropriate buffer conditions, and the presence of appropriate reagents. Appropriate temperature conditions may, in some cases, be such that each thermal cycle is performed at a desired annealing temperature. A desired annealing temperature may be sufficient for annealing of an oligonucleotide probe(s) to a nucleic acid target. Appropriate buffer conditions may, in some cases, be such that the appropriate salts are present in a buffer used during thermal cycling. Appropriate salts may include magnesium salts, potassium salts, ammonium salts. Appropriate buffer conditions may be such that the appropriate salts are present in appropriate concentrations. Appropriate reagents for amplification of each member of a plurality of nucleic acid targets with PCR may include deoxyribonucleotide triphosphates (dNTPs). dNTPs may comprise natural or non-natural dNTPs including, for example, dATP, dCTP, dGTP, dTTP, dUTP, and variants thereof.

In various aspects, primer extension reactions are utilized to generate amplified product. Primer extension reactions generally comprise a cycle of incubating a reaction mixture at a denaturation temperature for a denaturation duration and incubating a reaction mixture at an elongation temperature for an elongation duration. In any of the various aspects, multiple cycles of a primer extension reaction can be conducted. Any suitable number of cycles may be conducted. For example, the number of cycles conducted may be less than about 100, 90, 80, 70, 60, 50, 40, 30, 20, 10, or 5 cycles. The number of cycles conducted may depend upon, for example, the number of cycles (e.g., cycle threshold value (Ct)) used to obtain a detectable amplified product (e.g., a detectable amount of amplified DNA product that is indicative of the presence of a target DNA in a nucleic acid sample). For example, the number of cycles used to obtain a detectable amplified product (e.g., a detectable amount of DNA product that is indicative of the presence of a target DNA in a nucleic acid sample) may be less than about or about 100 cycles, 75 cycles, 70 cycles, 65 cycles, 60 cycles, 55 cycles, 50 cycles, 40 cycles, 35 cycles, 30 cycles, 25 cycles, 20 cycles, 15 cycles, 10 cycles, or 5 cycles. Moreover, in some embodiments, a detectable amount of an amplifiable product (e.g., a detectable amount of DNA product that is indicative of the presence of a target DNA in a nucleic acid sample) may be obtained at a cycle threshold value (Ct) of less than 100, 75, 70, 65, 60, 55, 50, 45, 40, 35, 30, 25, 20, 15, 10, or 5.

The time for which an amplification reaction yields a detectable amount of amplified nucleic acid may vary depending upon the nucleic acid sample, the sequence of the target nucleic acid, the sequence of the primers, the particular nucleic acid amplification reactions conducted, and the particular number of cycles of the amplification, the temperature of the reaction, the pH of the reaction. For example, amplification of a target nucleic acid may yield a detectable amount of product indicative to the presence of the target nucleic acid at time period of 120 minutes or less; 90 minutes or less; 60 minutes or less; 50 minutes or less; 45 minutes or less; 40 minutes or less; 35 minutes or less; 30 minutes or less; 25 minutes or less; 20 minutes or less; 15 minutes or less; 10 minutes or less; or 5 minutes or less.

In some embodiments, amplification of a nucleic acid may yield a detectable amount of amplified DNA at time period of 120 minutes or less; 90 minutes or less; 60 minutes or less; 50 minutes or less; 45 minutes or less; 40 minutes or less; 35 minutes or less; 30 minutes or less; 25 minutes or less; 20 minutes or less; 15 minutes or less; 10 minutes or less; or 5 minutes or less.

### Nucleic Acid Targets

A nucleic acid target of the present disclosure may be derived from a sample. A biological sample may be a sample derived from a subject. A sample may comprise any number of macromolecules, for example, cellular macromolecules. A sample may comprise a plurality of cells. A sample may comprises a plurality of viruses or microbes. A sample may be a tissue sample, such as a biopsy, core biopsy, needle aspirate, or fine needle aspirate. A sample may be formalin-fixed, paraffin-embedded (FFPE) tissue sample. A sample may be a tumor sample. A sample may be a fluid sample, such as a blood sample, plasma sample, urine sample, or saliva sample. A sample may be a skin sample. A biological sample may be a cheek swab. A sample may be a plasma or serum sample. A sample may comprise one or more cells. The one or more cells may be derived from a tumor. A biological sample may be, for example, blood, plasma, serum, urine, saliva, mucosal excretions, sputum, stool or tears. The sample may be obtained or derived from an environmental sample. For example, the sample may be a water sample or soil sample, or other samples found outside of a subject's body. The sample may be a sample derived from a pregnant subject. The sample may comprise fetal nucleic acids. The sample may comprise maternal nucleic acids. The sample may be a wastewater sample. The sample may be a collection of samples. For example, a sample may be pooled with other sample and then subjected to methods described elsewhere herein.

A nucleic acid target may be derived from one or more cells. A nucleic acid target may comprise deoxyribonucleic acid (DNA). DNA may be any kind of DNA, including genomic DNA. A nucleic acid target may be viral DNA. A nucleic acid target may comprise ribonucleic acid (RNA). RNA may be any kind of RNA, including messenger RNA, transfer RNA, ribosomal RNA, and microRNA. RNA may be viral RNA. The nucleic acids may comprise a human genomic sequence. The nucleic acids may comprise an animal genomic sequence. The nucleic acids may comprise a plant genomic sequence. The nucleic acids may comprise a fungal genomic sequence. The nucleic acids may comprise an archaeal genomic sequence. The nucleic acids may comprise a pathogen associated sequence. The nucleic acid may comprise a wild type sequence. The nucleic acid may comprise a variant sequence.

Nucleic acid targets may comprise one or more members. A member may be any region of a nucleic acid target. A member may be of any length. A member may be, for example, up to 1, 2, 3, 4, 5, 10, 20, 50, 100, 500, 1000, 5000, 10000, 50000, or 100000 nucleotides, or more. In some instances, a member may be a gene. A nucleic acid target may comprise a gene whose detection may be useful in diagnosing one or more diseases. The disease may be cancer. A gene may be a viral gene or bacterial gene whose detection may be useful in identifying the presence or absence of a pathogen in a subject. In some cases, the methods of the present disclosure are useful in detecting the presence or absence or one or more infectious agents (e.g., viruses, bacteria, fungi) in a subject. The nucleic acid targets may be a human gene. The nucleic acid targets may be associated with a disease, such as cancer. The nucleic acid target may be a nucleic acid derived from an infectious agent. For example, the nucleic acid target may comprise a sequence of an influenza gene. The influenza gene may be an influenza A PB gene, an influenza HA gene, or an influenza B NS gene. For example, the nucleic acid target may comprise a sequence of a SARS-COV-2 gene. The nucleic acid target may comprise a sequence that is indicative of the presence of the flu or COVID-19 in a subject. The SARS-COV-2 gene may be a SARS-COV-2 N gene or a SARS-COV-2 N gene. The SARS-COV-2 N gene may be a N1, N2 or N3 gene. The nucleic acid target may comprise a sequence indicative of a serotype. For example, the nucleic acid target may be an influenza HA gene sequence corresponding to the H1 or H3 serotype. The nucleic acid target may comprise a single nucleotide polymorphism (SNP). The nucleic acid target may allow a genotype to be determined. The nucleic acid target may be a region of the human genome that indicates a predisposition for a particular disease. For example, a particular mutation or SNP of in a subject may be associated with an increased risk of infection of a particular pathogen. For example, the detection of both a pathogenic nucleic acid sequence and the presence of the mutation in the subject's genome may indicate the subject is at a high risk.

Markers for a predisposition for a particular disease may be detected by assays as disclosed elsewhere herein. The markers for predisposition may be related to other existing conditions, for example, another disease such as diabetes, cancer, heart disease, or a condition that results in the individual being immune compromised. The markers may be related to immune response, for example, markers that indicate an intensity of a response to an antigen. The markers may be related to specific mutations or SNPs that are associated with higher infections. For example, a SNP of a receptor may have a higher affinity to a virus, thereby allowing the virus to more easily recognize and infect a cell.

Nucleic acid targets may be of various concentrations in the reaction. The nucleic acid sample may be diluted or concentrated to achieve different concentrations of nucleic acids. The concentration of the nucleic acids in the nucleic acid sample may at least 0.1 nanograms per microliter (ng/μL), 0.2 ng/μL, 0.5 ng/μL, 1 ng/μL, 2 ng/μL, 3 ng/μL, 5 ng/μL, 10 ng/μL, 20 ng/μL, 30 ng/μL, 40, ng/μL, 50 ng/μL, 100 ng/μL, 1000 ng/μL, 10000 ng/μL or more. In some cases, the concentration of the nucleic acids in the nucleic acid sample may be at most ng/μL, 0.2 ng/μL, 0.5 ng/μL, 1 ng/μL, 2 ng/μL, 3 ng/μL, 5 ng/μL, 10 ng/μL, 20 ng/μL, 30 ng/μL, 40, ng/μL, 50 ng/μL, 100 ng/μL, 1000 ng/μL, 10000 ng/μL or less.

### Sample Processing

A sample may be processed concurrently with, prior to, or subsequent to the methods of the present disclosure. A sample may be processed to purify or enrich for nucleic acids (e.g., to purify nucleic acids from a plasma sample). A sample comprising nucleic acids may be processed to purity or enrich for nucleic acid of interest. A sample may undergo an extraction to extract molecules used in the assay. For example, the extraction may use a column to bind or interact with a molecule. For example, an RNA extraction kit may be used such as a Qiagen RNA mini kit to extract or isolate RNA. A sample may be not processed after being added to a sample collection tube. A sample may not undergo any processing that purifies or enriches for nucleic acids and the assay may be performed directly on the sample. A sample may not require processing to purify or enrich for nucleic acids. The assay may have a sensitivity such that a sample that does not undergo processing may be assayed and result in an accurate result. A sample may not undergo an extraction reaction such to extract nucleic acids after the sample is received and prior to running an assay. A sample may not undergo an extraction reaction such to purify nucleic acids after the sample is received and prior to running an assay. A sample may be diluted. A sample may be diluted at least at 1:1, 1:2, 1:3, 1:4, 1:5, 1:6, 1:7, 1:8, 1:9, 1:10, 1:11, 1:12, 1:13, 1:14, 1:15: 1:16: 1:17, 1:18, 1:19, 1:20, 1:30, 1:40, 1:50, 1:60, 1:70, 1:80, 1:90, 1:100, or 1:1000, 1:10000, 1:100000 or more. A sample may be diluted at no more than at 1:1, 1:2, 1:3, 1:4, 1:5, 1:6, 1:7, 1:8, 1:9, 1:10, 1:11, 1:12, 1:13, 1:14, 1:15: 1:16: 1:17, 1:18, 1:19, 1:20, 1:30, 1:40, 1:50, 1:60, 1:70, 1:80, 1:90, 1:100, or 1:1000, 1:0000, 1:100000, or less. The sample may be diluted in a buffer or a solution. For example, the sample may be diluted in Tris-Ethylenediaminetetraacetic acid (TE) buffer. The sample may be diluted with a solution comprising alcohol. The sample may be diluted with a solution comprising sodium acetate.

The assays, methods, and systems of the present disclosure may be able to detect a nucleic acid target in a sample without sample processing steps. For example, the detection may be performed on a sample that has not been purified or enriched for a nucleic acid. An assay may take a sample comprising nucleic acids from a subject, subject the nucleic acids to a reverse transcriptase and polymerase chain reaction directly, without a step or purification or enrichment, and detect the nucleic acid target. An assay may, for example, dilute a sample comprising nucleic acids from a subject into a suitable buffer, subject the nucleic acids to a reverse transcriptase and polymerase chain reaction directly, without a step or purification or enrichment, and detect the nucleic acid target.

### Nucleic Acid Enzymes

Mixtures and compositions of the present disclosure may comprise one or more nucleic acid enzymes. A nucleic acid enzyme may have exonuclease activity. A nucleic acid enzyme may have endonuclease activity. A nucleic acid enzyme may have RNase activity. A nucleic acid enzyme may be capable of degrading a nucleic acid comprising one or more ribonucleotide bases. A nucleic acid enzyme may be, for example, RNase H or RNase III. An RNase III may be, for example, Dicer. A nucleic acid may be an endonuclease I such as, for example, a T7 endonuclease I. A nucleic acid enzyme may be capable of degrading a nucleic acid comprising a non-natural nucleotide. A nucleic acid enzyme may be an endonuclease V such as, for example, an E. coli endonuclease V.

A nucleic acid enzyme may be a polymerase (e.g., a DNA polymerase). A DNA polymerase may be used. Any suitable DNA polymerase may be used, including commercially available DNA polymerases. A DNA polymerase generally refers to an enzyme that is capable of incorporating nucleotides to a strand of DNA in a template bound fashion. A polymerase may be Taq polymerase or a variant thereof. Non-limiting examples of DNA polymerases include Taq polymerase, Tth polymerase, Tli polymerase, Pfu polymerase, VENT polymerase, DEEPVENT polymerase, EX-Taq polymerase, LA-Taq polymerase, Expand polymerases, Sso polymerase, Poc polymerase, Pab polymerase, Mth polymerase, Pho polymerase, ES4 polymerase, Tru polymerase, Tac polymerase, Tne polymerase, Tma polymerase, Tih polymerase, Tfi polymerase, Platinum Taq polymerases, Hi-Fi polymerase, Tbr polymerase, Tfl polymerase, Pfutubo polymerase, Pyrobest polymerase, Pwo polymerase, KOD polymerase, Bst polymerase, Sac polymerase, Klenow fragment, and variants, modified products and derivatives thereof. For certain Hot Start Polymerase, a denaturation step at 94° C.-95° C. for 2 minutes to 10 minutes may be required, which may change the thermal profile based on different polymerases. A nucleic acid enzyme may be capable, under appropriate conditions, of degrading an oligonucleotide probe. For example, a nucleic acid enzyme may be a polymerase and comprise exo activity and degrade a probe resulting in a detectable signal. A nucleic acid enzyme may be capable, under appropriate conditions, of releasing a quencher from an oligonucleotide probe.

### Reactions

In various aspects disclosed elsewhere herein, reactions are performed. A reaction may comprise contacting nucleic acid targets with one or more oligonucleotide probes. A reaction may comprise contacting a sample solution volume (e.g., a droplet, well, tube, etc.) with a plurality of oligonucleotide probes, each corresponding to one of a plurality of nucleic acid targets, to generate a plurality of signals generated from the plurality of oligonucleotide probes. A reaction may comprise polymerase chain reaction (PCR).

### Oligonucleotide Primers

In various aspects disclosed elsewhere herein, oligonucleotide primers are used. An oligonucleotide primer (or “amplification oligomer”) of the present disclosure may be a deoxyribonucleic acid. An oligonucleotide primer may be a ribonucleic acid. An oligonucleotide primer may comprise one or more non-natural nucleotides. A non-natural nucleotide may be, for example, deoxyinosine. The oligonucleotide primer may be able to hybridize to a first analyte and a second analyte, and may generates a first signal corresponding to said first analyte and a second signal corresponding to said second analytes.

An oligonucleotide primer may be a forward primer. An oligonucleotide primer may be a reverse primer. An oligonucleotide primer may be between about 5 and about 50 nucleotides in length. An oligonucleotide primer may be at least 5, 10, 11, 12, 13, 14, 15, 16, 17, 18, 19, 20, 25, 30, 35, 40, 45, or 50 base pairs in length, or more. An oligonucleotide primer may be at most 50, 45, 40, 35, 30, 25, 20, 19, 18, 17, 16, 15, 14, 13, 12, 11, 10, or 5 nucleotides in length. An oligonucleotide primer may be about 5, 10, 11, 12, 13, 14, 15, 16, 17, 18, 19, 20, 25, 30, 35, 40, 45, or 50 base pairs in length.

A set of oligonucleotide primers may comprise paired oligonucleotide primers. Paired oligonucleotide primers may comprise a forward oligonucleotide primer and a reverse oligonucleotide primer. A forward oligonucleotide primer may be configured to hybridize to a first region (e.g., a 3′ end) of a nucleic acid sequence, and a reverse oligonucleotide primer may be configured to hybridize to a second region (e.g., a 5′ end) of the nucleic acid sequence, thereby being configured to amplify the nucleic acid sequence under conditions sufficient for nucleic acid amplification. Different sets of oligonucleotide primers may be configured to amplify different nucleic acid target sequences. For example, a first set of oligonucleotide primers may be configured to amplify a first nucleic acid sequence of a given length, and a second set of oligonucleotide primers may be configured to amplify a second nucleic acid sequence of shorter length than the first nucleic acid sequence. In another example, a first set of oligonucleotide primers may be configured to amplify a first nucleic acid sequence of a given length, and a second set of oligonucleotide primers may be configured to amplify a second nucleic acid sequence of longer length than the first nucleic acid sequence.

A primer may comprise a mismatch to any member of a plurality of nucleic acids. A primer may comprise less than 100% complementarity to any member of a plurality of nucleic acids. A primer may be 100% complementary to any member of a plurality of nucleic acids. The primer may comprise a mismatch to a first analyte in a sample and may be 100% complementary to a second analyte in a sample. A primer with less than 100% complementarity to a first analyte may result in reduced efficacy of amplification as compared to the amplification of a second analyte with 100% complementarity to the primer. The reduced efficiency may generate a different kinetic signature as compared to a kinetic signature for an analyte without reduced amplification efficiency. Comparing kinetic signatures may enable quantification of analytes by kinetic signature analysis.

A mixture may comprise a plurality of forward oligonucleotide primers. A plurality of forward oligonucleotide primers may be a deoxyribonucleic acid. Alternatively, a plurality of forward oligonucleotide primers may be a ribonucleic acid. A plurality of forward oligonucleotide primers may be between about 5 and about 50 nucleotides in length. A plurality of forward oligonucleotide primer may be at least 5, 10, 11, 12, 13, 14, 15, 16, 17, 18, 19, 20, 25, 30, 35, 40, 45, or 50 base pairs in length, or more. A plurality of forward oligonucleotide primer may be at most 50, 45, 40, 35, 30, 25, 20, 19, 18, 17, 16, 15, 14, 13, 12, 11, 10, or 5 nucleotides in length.

A mixture may comprise a plurality of reverse oligonucleotide primers. A plurality of reverse oligonucleotide primers may be a deoxyribonucleic acid. Alternatively, a plurality of reverse oligonucleotide primers may be a ribonucleic acid. A plurality of reverse oligonucleotide primers may be between about 5 and about 50 nucleotides in length. A plurality of reverse oligonucleotide primer may be at least 5, 10, 11, 12, 13, 14, 15, 16, 17, 18, 19, 20, 25, 30, 35, 40, 45, or 50 base pairs in length, or more. A plurality of reverse oligonucleotide primer may be at most 50, 45, 40, 35, 30, 25, 20, 19, 18, 17, 16, 15, 14, 13, 12, 11, 10, or 5 nucleotides in length.

A set of oligonucleotide primers (e.g., a forward primer and a reverse primer) may be configured to amplify a nucleic acid sequence of a given length (e.g., may hybridize to regions of a nucleic acid sequence a given distance apart). A pair of oligonucleotide primers may be configured to amplify a nucleic acid sequence of a length of at least 50, at least 75, at least 100, at least 125, at least 150, at least 175, at least 200, at least 225, at least 250, at least 275, or at least 300 base pairs (bp), or more. A pair of oligonucleotide primers may be configured to amplify a nucleic acid sequence of a length of at most 300, at most 275, at most 250, at most 225, at most 200, at most 175, at most 150, at most 125, at most 100, at most 75, or at most 50 bp, or less.

In some aspects, the primer may be configured to amplify sequences derived from influenza virus, coronavirus, respiratory syncytial virus, hepatitis virus, herpesvirus, or papillomavirus. The primer may be configured to amplify a SARS-COV-2 N gene. The primer may be configured to amplify a SARS-COV-2 E gene. The primer may be configured to amplify an influenza A PB1 gene, influenza A H1 gene, influenza B NS gene.

In some aspects, the primer may be configured to hybridize, anneal or be homologous to sequences derived from humans. The sequence may be a sequence associated with cancer. The sequence may be associated with trisomy or fetal abnormalities.

In some aspects, a mixture may include one or more synthetic (or otherwise generated to be different from the target of interest) primers for PCR reactions.

In some aspects, a mixture may be subjected to conditions sufficient to anneal an oligonucleotide primer to a nucleic acid molecule. In some aspects, a mixture may be subjected to conditions sufficient to anneal a plurality of oligonucleotide primers to a nucleic acid molecule.

In some aspects, a mixture may be subjected to conditions sufficient to anneal a plurality of oligonucleotide primers to a plurality of nucleic acid targets. The mixture may be subjected to conditions which are sufficient to denature nucleic acid molecules. Subjecting a mixture to conditions sufficient to anneal an oligonucleotide primer to a nucleic acid target may comprise thermally cycling the mixture under reaction conditions appropriate to amplify the nucleic acid target(s) with, for example, polymerase chain reaction (PCR).

Conditions may be such that an oligonucleotide primer pair (e.g., forward oligonucleotide primer and reverse oligonucleotide primer) are degraded by a nucleic acid enzyme. An oligonucleotide primer pair may be degraded by the exonuclease activity of a nucleic acid enzyme. An oligonucleotide primer pair may be degraded by the RNase activity of a nucleic acid enzyme. Degradation of the oligonucleotide primer pair may result in release of the oligonucleotide primer. Once released, the oligonucleotide primer pair may bind or anneal to a template nucleic acid.

### Oligonucleotide Probes

In various aspects disclosed elsewhere herein, oligonucleotide probes are used. Samples, mixtures, kits, and compositions of the present disclosure may comprise an oligonucleotide probe, also referenced herein as a “detection probe” or “probe”. An oligonucleotide probe may be a nucleic acid (e.g., DNA, RNA, etc.). An oligonucleotide probe may comprise a region complementary to a region of a nucleic acid target. The concentration of an oligonucleotide probe may be such that it is in excess relative to other components in a sample.

The oligonucleotide probe may be able to hybridize to a first analyte and a second analyte, and may generates a first signal corresponding to said first analyte and a second signal corresponding to said second analytes.

An oligonucleotide probe may comprise a non-target-hybridizing sequence. A non-target-hybridizing sequence may be a sequence which is not complementary to any region of a nucleic acid target sequence. An oligonucleotide probe comprising a non-target-hybridizing sequence may be a hairpin detection probe. An oligonucleotide probe comprising a non-target-hybridizing sequence may be a molecular beacon probe. Examples of molecular beacon probes are provided in, for example, U.S. Pat. No. 7,671,184, incorporated herein by reference in its entirety. An oligonucleotide probe comprising a non-target-hybridizing sequence may be a molecular torch. Examples of molecular torches are provided in, for example, U.S. Pat. No. 6,534,274, incorporated herein by reference in its entirety.

A sample may comprise more than one oligonucleotide probe. Multiple oligonucleotide probes may be the same or may be different. An oligonucleotide probe may be at least 5, at least 10, at least 15, at least 20, or at least 30 nucleotides in length, or more. An oligonucleotide probe may be at most 30, at most 20, at most 15, at most 10 or at most 5 nucleotides in length. In some examples, a mixture comprises a first oligonucleotide probe and one or more additional oligonucleotide probes. An oligonucleotide probe may be a nucleic acid (e.g., DNA, RNA, etc.). An oligonucleotide probe may be at least 2, 3, 4, 5, 6, 7, 8, 9, 10, 20, 30, 40, or 50 nucleotides in length, or more. An oligonucleotide probe may be at most 50, 40, 30, 20, 10, 9, 8, 7, 6, 5, 4, 3, or 2 nucleotides in length.

In some cases, 2, 3, 4, 5, 6, 7, 8, 9, 10, 11, 12, 13, 14, 15, 20, or more different oligonucleotide probes may be used. Each oligonucleotide probe may correspond to (e.g., capable of binding to) a given region of a nucleic acid target (e.g., a chromosome) in a sample. In one example, a first oligonucleotide probe is specific for a first region of a first nucleic acid target, a second oligonucleotide probe is specific for a second region of the first nucleic acid target, and a third oligonucleotide probe is specific for a third region of the first nucleic acid target. Each oligonucleotide probe may comprise a signal tag with about equal emission wavelengths. In some cases, each oligonucleotide probe comprises an identical fluorophore. In some cases, each oligonucleotide probe comprises a different fluorophore. In some case, each fluorophore is capable of being detected in a single optical channel. In other case, a fluorophore may be detected in multiple channels. In some cases, an oligonucleotide probe may have similar or the same detectable agent or fluorophore as another oligonucleotide probe in the sample. In some cases, an oligonucleotide probe may have a different detectable agent or fluorophore as compared to another oligonucleotide probe in the sample. In some cases, an oligonucleotide probe may have similar sequence or be capable or binding a similar sequence as another oligonucleotide probe in the sample. In some cases, an oligonucleotide probe may have a different sequence or be capable of binding a different sequence as compared to another oligonucleotide probe in the sample.

A probe may correspond to a region of a nucleic acid target. For example, a probe may have complementarity and/or homology to a region of a nucleic acid target. A probe may comprise a region which is complementary or homologous to a region of a nucleic acid target. A probe corresponding to a region of a nucleic acid target may be capable of binding to the region of the nucleic acid target under appropriate conditions (e.g., temperature conditions, buffer conditions. etc.). For example, a probe may be capable of binding to a region of a nucleic acid target under conditions appropriate for polymerase chain reaction. A probe may correspond to an oligonucleotide which corresponds to a nucleic acid target. For example, an oligonucleotide may be a primer with a region complementary to a nucleic acid target and a region complementary to a probe.

In some aspects, the probe may be configured to hybridize, anneal or be homologous to sequences derived from a virus. For example the virus may comprise an influenza virus, coronavirus, respiratory syncytial virus, hepatitis virus, herpesvirus, or papillomavirus. The probe may be configured to hybridize, anneal or be homologous to sequences of a SARS-COV-2 N gene. The probe may be configured to amplify a SARS-COV-2 E gene. The probe may be configured to hybridize, anneal or be homologous to sequences of an influenza A PB1 gene, influenza A H1 gene, influenza B NS gene.

In some aspects, the probe may be configured to hybridize, anneal or be homologous to sequences derived from humans. The sequence may be a sequence associated with cancer. The sequence may be associated with trisomy or fetal abnormalities.

A probe may be provided at a specific concentration. In some cases, a second nucleic acid probe is provided at a concentration of at least about 2×, about 3×, about 4×, about 5×, about 6×, about 7×, about 8×, or more. In some cases, a second nucleic acid probe is provided at a concentration of at most about 8×, about 7×, about 6×, about 5×, about 4×, about 3×, or about 2×. In some cases, a second nucleic acid probe is provided at a concentration of about 2×, about 3×, about 4×, about 5×, about 6×, about 7×, or about 8×. × may be a concentration of a nucleic acid probe provided in the disclosed methods. In some cases, × is at least 50 nM, 100 nM, 150 nM, 200 nM, 250 nM, 300 nM, 350 nM, 400 nM, 450 nM, 500 nM, or greater. In some cases, × is at most 500 nM, 450 nM, 400 nM, 350 nM, 300 nM, 250 nM, 200 nM, 150 nM, 100 nM, or 50 nM. × may be any concentration of a nucleic acid probe.

A probe may be a nucleic acid complementary to a region of a given nucleic acid target. Each probe used in the methods and assays of the presence disclosure may comprise at least one fluorophore. A fluorophore may be selected from any number of fluorophores. A fluorophore may be selected from three, four, five, six, seven, eight, nine, or ten fluorophores, or more. One or more oligonucleotide probes used in a single reaction may comprise the same fluorophore. In some cases, all oligonucleotide probes used in a single reaction comprise the same fluorophore. Each probe may, when excited and contacted with its corresponding nucleic acid target, generate a signal. A signal may be a fluorescent signal. A plurality of signals may be generated from one or more probes.

An oligonucleotide probe may have less than 50%, 40%, 30%, 20%, 10%, 5%, or 1% complementarity to any member of a plurality of nucleic acid targets. An oligonucleotide probe may have no complementarity to any member of the plurality of nucleic acid targets.

A probe may comprise a mismatch to any member of a plurality of nucleic acids. The probe may be 100% complementary to any member of a plurality of nucleic acids. The probe may comprise a mismatch to a first analyte in a sample and may be 100% complementary to a second analyte in a sample.

An oligonucleotide probe may comprise a detectable label. A detectable label may be a chemiluminescent label. A detectable label may comprise a fluorescent label. A detectable label may comprise a fluorophore. A fluorophore may be, for example, FAM, TET, HEX, JOE, Cy3, or Cy5. A fluorophore may be FAM. A fluorophore may be HEX. An oligonucleotide probe may further comprise one or more quenchers. A quencher may inhibit signal generation from a fluorophore. A quencher may be, for example, TAMRA, BHQ-1, BHQ-2, or Dabcy. A quencher may be BHQ-1. A quencher may be BHQ-2.

### Signal Generation

Thermal cycling may be performed such that one or more oligonucleotide probes are degraded by a nucleic acid enzyme. An oligonucleotide probe may be degraded by the exonuclease activity of a nucleic acid enzyme. An oligonucleotide probe may generate a signal upon degradation. In some cases, an oligonucleotide probe may generate a signal only if at least one member of a plurality of nucleic acid targets is present in a mixture.

In various aspects, extension reactions and amplification reactions may be used to allow for the generation of signals. The extension reaction and amplification reaction may be used to generate a signal correspond to an analyte. The extension reaction may extend an oligonucleotide that can hybridize to more than one analyte. Based on the hybridization partner, the extension reaction may generate a different signal. Extension or amplification of a first analytes may generate a first signal whereas the extension or amplification of a second analyte may generate a second signal. The efficiency of the hybridization reactions may affect the extension reaction or the generation of a signal. The extension or amplification reaction may generate a signal by degrading or reaction with the oligonucleotide that can hybridize to more than one analyte. The oligonucleotide that can hybridize to more than one analyte may be a probe, and the extension or amplification reaction may allow generation of a signal from the probe. The probe may hybridize with different efficiency or affinity and may allow the generation of a different signal based on the analyte hybridized thereto. For example, a probe may bind less efficiently to a first analyte and may result in generation of a lower intensity of signal as compared to the binding and signal intensity when bound to a second analyte.

Signal generation may correspond to reactions conditions of reactions relating to signal generation. The signal generation may be altered by a hybridization efficiency of the oligonucleotide. For example, an oligonucleotide may have a hybridization efficiency to a first analyte and a different hybridization efficiency to a second analyte which may in turn affect the generation of signal or alter the resulting signal that is generated. In the case of amplification or extension reactions, a time period or temperature may be altered such to change the signal generation efficiency or a kinetic signature shape. For example, a sample may comprise more than one analyte and an oligonucleotide that can hybridize to more than one analyte may be added to the sample. The different signal generation efficiency or kinetic shape of a reaction may be used to differentiate a first analyte and a second analyte. The annealing temperature of a reaction may be altered such that the hybridization to one analyte is favored over the hybridization to another analyte. Multiple reactions may be performed at different annealing temperature (for example using a gradient) that allows for a signal to be generated and distinguishable for different analytes. The reactions may be performed such that a first reaction has a more stringent annealing condition compared to a second reaction. The reactions may comprise an annealing time, and the annealing time may be modulated to affect the generation of a signal. For example, a first reaction may comprise an annealing time that is longer than an annealing time for a second reaction. For example, a first reaction may comprise an annealing time that is shorter than an annealing time for a second reaction. Similarly, extension times and temperatures may be modulated to affect the generation of a signal and allow different signal to be obtained based on the analyte. For example, a first reaction may comprise an extension time that is longer than an extension time for a second reaction. For example, a first reaction may comprise an extension time that is shorter than an extension time for a second reaction. For example, a first reaction may comprise an extension temperature that is higher (or lower) than an extension temperature for a second reaction. For example, a first reaction may comprise an extension temperature that is lower than an extension temperature for a second reaction.

A reaction may generate one or more signals. A reaction may generate a cumulative intensity signal comprising a sum of multiple signals. A signal may be a chemiluminescent signal. A signal may be a fluorescent signal. A signal may be generated by an oligonucleotide probe. For example, excitation of a hybridization probe comprising a luminescent signal tag may generate a signal. A signal may be generated by a fluorophore. A fluorophore may generate a signal upon release from a hybridization probe. A reaction may comprise excitation of a fluorophore. A reaction may comprise signal detection. A reaction may comprise detecting emission from a fluorophore.

A signal may be a fluorescent signal. A signal may correspond to a fluorescence intensity level. Each signal measured in the methods of the present disclosure may have a distinct fluorescence intensity value, thereby corresponding to the presence of a unique combination of nucleic acid targets. A signal may be generated by one or more oligonucleotide probes. Multiple signals may be generated by an oligonucleotide probe. For example, a oligonucleotide may be able to bind to multiple analytes and may generate a signal corresponding to hybridization with a first analyte and a second signal corresponding with a second analyte.

N may be a number of signals detected in a single optical channel in an assay of the present disclosure. N may be at least 2, 3, 4, 5, 6, 7, 8, 9, 10, 11, 12, 13, 14, 15, 20, 25, 30, 40, 50 or more. N may be at most 50, 40, 30, 24, 20, 15, 14, 13, 12, 11, 10, 9, 8, 7, 6, 5, 4, 3, or 2. N may be 2, 3, 4, 5, 6, 7, 8, 9, 10, 11, 12, 13, 14, 15, 20, 25, 30, 40, or 50.

As will be recognized and is described elsewhere herein, sets of signals may be generated in multiple different optical channels, where each set of signals is detected in a single optical channel, thereby significantly increasing the number of nucleic acid targets that can be measured in a single reaction. In some cases, two sets of signals are detected in a single reaction. Each set of signals detected in a reaction may comprise the same number of signals, or different numbers of signals.

In some cases, a signal may be generated simultaneous with hybridization of an oligonucleotide probe to a region of a nucleic acid. For example, an oligonucleotide probe (e.g., a molecular beacon probe or molecular torch) may generate a signal (e.g., a fluorescent signal) following hybridization to a nucleic acid. In some cases, a signal may be generated subsequent to hybridization of an oligonucleotide probe to a region of a nucleic acid, following degradation of the oligonucleotide probe by a nucleic acid enzyme.

In cases where an oligonucleotide probe comprises a signal tag, the oligonucleotide probe may be degraded when bound to a region of an oligonucleotide primer, thereby generating a signal. For example, an oligonucleotide probe (e.g., a TaqMan® probe) may generate a signal following hybridization of the oligonucleotide probe to a nucleic acid and subsequent degradation by a polymerase (e.g., during amplification, such as PCR amplification). An oligonucleotide probe may be degraded by the exonuclease activity of a nucleic acid enzyme.

An oligonucleotide probe may comprise a quencher and a fluorophore, such that the quencher is released upon degradation of an oligonucleotide probe, thereby generating a fluorescent signal. Thermal cycling may be used to generate one or more signals. Thermal cycling may generate at least 1, 2, 3, 4, 5, 6, 7, 8, 9, 10 signals, or more. Thermal cycling may generate at most 10, 9, 8, 7, 6, 5, 4, 3, 2, or 1 signal. Multiple signals may be of the same type or of different types. Signals of different types may be fluorescent signals with different fluorescent wavelengths. Signals of different types may be generated by detectable labels comprising different fluorophores. Signals of the same type may be of different intensities (e.g., different intensities of the same fluorescent wavelength). Signals of the same type may be signals detectable in the same color channel. Signals of the same type may be generated by detectable labels comprising the same fluorophore. Detectable labels comprising the same fluorophore may generate different signals by nature of being at different concentrations, thereby generating different intensities of the same signal type.

Although fluorescent probes have been used to illustrate this principle, the disclosed methods are equally applicable to any other method providing a quantifiable signal, including an electrochemical signal, chemiluminescent signals, magnetic particles, and electrets structures exhibiting a permanent dipole.

In certain portions of this disclosure, the signal may be a fluorescent signal. For example, like fluorescent signals, any of the electromagnetic signals described above may also be characterized in terms of a wavelength, whereby the wavelength of a fluorescent signal may also be described in terms of color. The color may be determined based on measuring intensity at a particular wavelength or range of wavelengths, for example by determining a distribution of fluorescent intensity at different wavelengths and/or by utilizing a band pass filter to determine the fluorescence intensity within a particular range of wavelengths.

The presence or absence of one or more signals may be detected. One signal may be detected, or multiple signals may be detected. Multiple signals may be detected simultaneously. Alternatively, multiple signals may be detected sequentially. A signal may be detected throughout the process of thermal cycling, for example, at the end of each thermal cycle. The signals may be detected in a multi-channel detector. For example, the signal may be observed using a detector that can observe a signal in multiple ranges of wavelengths simultaneously, substantially simultaneously, or sequentially. The signal may be observable in at least 1, 2, 3, 4, 5, 6, 7, 8, 9, 10, or more channels. The signal may be observable in no more than 10, 9, 8, 7, 6, 5, 4, 3, 2, or less channels.

In some cases, the signal intensity increases with each thermal cycle. The signal intensity may increase in a sigmoidal fashion. The presence of a signal may be correlated to the presence of at least one member of a plurality of target nucleic acids. Correlating the presence of a signal to the presence of at least one member of a plurality of target nucleic acids may comprise establishing a signal intensity threshold. A signal intensity threshold may be different for different signals. Correlating the presence of a signal to the presence of at least one member of a plurality of target nucleic acids may comprise determining whether the intensity of a signal increases beyond a signal intensity threshold. In some examples, the presence of a signal may be correlated with the presence of at least one of all members of a plurality of target nucleic acids. In other examples, the presence of a first signal may be correlated with the presence of at least one of a first subset of members of a plurality of target nucleic acids, and the presence of a second signal may be correlated with the presence of at least one of a second subset of members of a plurality of target nucleic acids.

The presence of a signal may be correlated to the presence of a nucleic acid target. The presence of least 1, 2, 3, 4, 5, 6, 7, 8, 9, 10, or more signals may be correlated with the presence of at least one of at least 1, 2, 3, 4, 5, 6, 7, 8, 9, 10, or more nucleic acid targets. The absence of a signal may be correlated with the absence of corresponding nucleic acid targets. The absence of least 1, 2, 3, 4, 5, 6, 7, 8, 9, 10, or more signals may be correlated with the absence of each of at least 1, 2, 3, 4, 5, 6, 7, 8, 9, 10, or more nucleic acid target molecules. The presence of a plurality of signals may be correlated with a combination of targets. The presence of a plurality of signals may be correlated with a unique combination of targets. For example, the detection of a particular plurality of signals may indicate the presence or absence of a unique or particular combination of targets.

### Kits

The present disclosure provides kits for sample collection. The kit may comprise a sample collection vessel or sample collection tube. The kit may comprise a sample collection tool or an object that can obtain a sample via the contact of cells or nucleic acids from the subject and transfer sample to a sample collection vessel or tube. The sample collection tool may comprise a swab.

The present disclosure also provides kits for performing assays or analysis of assay results. Kits may comprise one or more oligonucleotide probes. Oligonucleotide probes may be lyophilized. Different oligonucleotide probes may be present at different concentrations in a kit. Oligonucleotide probes may comprise a fluorophore and/or one or more quenchers.

Kits may comprise one or more sets of oligonucleotide primers or oligonucleotide probes as described herein. The kit may comprise (i) a primer oligonucleotide configured to hybridize to said first analyte and said second analyte, wherein said oligonucleotide generates a first signal corresponding to said first analyte and a second signal corresponding to said second signal and (ii) a probe oligonucleotide configured to bind to said first analyte or said second analyte. A kit may comprise (i) a probe oligonucleotide configured to hybridize to said first analyte and said second analyte, wherein said probe oligonucleotide generates a first signal corresponding to said first analyte and a second signal corresponding to said second signal and (ii) a set of primer oligonucleotide configured to bind to said first analyte or said second analyte. The kits may further comprise a set of oligonucleotide primers comprising paired oligonucleotide primers. Paired oligonucleotide primers may comprise a forward oligonucleotide primer and a reverse oligonucleotide primer. A set of oligonucleotide primers may be configured to amplify a nucleic acid sequence corresponding to particular targets. For example, a forward oligonucleotide primer may be configured to hybridize to a first region (e.g., a 3′ end) of a nucleic acid sequence, and a reverse oligonucleotide primer may be configured to hybridize to a second region (e.g., a 5′ end) of the nucleic acid sequence, thereby being configured to amplify the nucleic acid sequence. Different sets of oligonucleotide primers may be configured to amplify nucleic acid sequences. In one example, a first set of oligonucleotide primers may be configured to amplify a first nucleic acid sequence, and a second set of oligonucleotide primers may be configured to amplify a second nucleic acid sequence. Oligonucleotide primers configured to amplify nucleic acid molecules may be used in performing the disclosed methods. In some cases, all of the oligonucleotide primers in a kit are lyophilized.

Kits may comprise one or more nucleic acid enzymes. A nucleic acid enzyme may be a nucleic acid polymerase. A nucleic acid polymerase may be a deoxyribonucleic acid polymerase (DNase). A DNase may be a Taq polymerase or variant thereof. A nucleic acid enzyme may be a ribonucleic acid polymerase (RNase). An RNase may be an RNase III. An RNase III may be Dicer. The nucleic acid enzyme may be an endonuclease. An endonuclease may be an endonuclease I. An endonuclease I may be a T7 endonuclease I. A nucleic acid enzyme may be capable of degrading a nucleic acid comprising a non-natural nucleotide. A nucleic acid enzyme may be an endonuclease V such as, for example, an E. coli endonuclease V. A nucleic acid enzyme may be a polymerase (e.g., a DNA polymerase). A polymerase may be Taq polymerase or a variant thereof. A nucleic acid enzyme may be capable, under appropriate conditions, of degrading an oligonucleotide probe. A nucleic acid enzyme may be capable, under appropriate conditions, of releasing a quencher from an oligonucleotide probe. Kits may comprise instructions for using any of the foregoing in the methods described herein.

### Systems

Methods as disclosed herein may be performed using a variety of systems. The systems may be configured such the steps of the method may be performed. For example, the systems may comprise a detector for the detection of signals as described elsewhere herein. The system may comprise a processor configured to process, receive, plot, or otherwise represent the data obtained from the detector. The processor may be configured to process the data as described elsewhere herein. The processor may be configured to generate a report of the results obtained from the assay. The results of the assay may be uploaded into a remote server, or other computer systems as described elsewhere herein. The results may be uploaded and sent to a subject's medical provider or an institution monitoring the spread of a disease. The results may also be sent to the subject directly. The subject, medical provider, or other institution may be able to access the remote server such review or analyze the results. For example, the results may then be transmitted to another institution/or medical professional for monitoring or for providing recommendations for the subject. In additional to the data generated for the detection of targets, the data may be used to monitor a geographical location of the assay or subject, for example to allow monitoring of the transmission of a disease. These results can then be uploaded into a cloud database or other remote database for storage and transmission to or access by a variety or individuals and institutions which may use the results of the assay. The results may be obtained on a smart phone or other computer system as disclosed elsewhere herein which may display the results.

The present disclosure provides computer systems that are programmed to implement methods of the disclosure. The computer system can perform various aspects of the present disclosure. The computer system can be an electronic device of a user or a computer system that is remotely located with respect to the electronic device. The electronic device can be a mobile electronic device.

The computer system may include a central processing unit (CPU, also “processor” and “computer processor” herein), which can be a single core or multi core processor, or a plurality of processors for parallel processing. The computer system may include memory or memory location (e.g., random-access memory, read-only memory, flash memory), electronic storage unit (e.g., hard disk), communication interface (e.g., network adapter) for communicating with one or more other systems, and peripheral devices, such as cache, other memory, data storage and/or electronic display adapters. The memory, storage unit, interface and peripheral devices are in communication with the CPU through a communication bus (solid lines), such as a motherboard. The storage unit can be a data storage unit (or data repository) for storing data. The computer system can be operatively coupled to a computer network (“network”) with the aid of the communication interface. The network can be the Internet, an internet and/or extranet, or an intranet and/or extranet that is in communication with the Internet. The network in some cases is a telecommunication and/or data network. The network can include one or more computer servers, which can enable distributed computing, such as cloud computing. The network, in some cases with the aid of the computer system, can implement a peer-to-peer network, which may enable devices coupled to the computer system to behave as a client or a server.

The CPU can execute a sequence of machine-readable instructions, which can be embodied in a program or software. The instructions may be stored in a memory location, such as the memory. The instructions can be directed to the CPU, which can subsequently program or otherwise configure the CPU to implement methods of the present disclosure. Examples of operations performed by the CPU can include fetch, decode, execute, and writeback.

The CPU can be part of a circuit, such as an integrated circuit. One or more other components of the system can be included in the circuit. In some cases, the circuit is an application specific integrated circuit (ASIC).

The storage unit can store files, such as drivers, libraries and saved programs. The storage unit can store user data, e.g., user preferences and user programs, or raw data or processed results from the assays. The computer system in some cases can include one or more additional data storage units that are external to the computer system, such as located on a remote server that is in communication with the computer system through an intranet or the Internet.

The computer system can communicate with one or more remote computer systems through the network. For instance, the computer system can communicate with a remote computer system of a user (e.g., operator). Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system via the network.

Methods as described herein can be implemented by way of machine (e.g., computer processor) executable code stored on an electronic storage location of the computer system, such as, for example, on the memory or electronic storage unit. The machine executable or machine-readable code can be provided in the form of software. During use, the code can be executed by the processor. In some cases, the code can be retrieved from the storage unit and stored on the memory for ready access by the processor. In some situations, the electronic storage unit can be precluded, and machine-executable instructions are stored on memory.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system can include or be in communication with an electronic display that comprises a user interface (UI) for providing, for example, plots of data, plots of kinetic signatures, information relating to signal amplitude, Examples of UIs include, without limitation, a graphical user interface (GUI) and web-based user interface.

Methods and systems of the present disclosure can be implemented by way of one or more algorithms. An algorithm can be implemented by way of software upon execution by the central processing unit. The algorithm can, for example, parameterize data points or fit data point to specified mathematical functions, in order to quantify analytes.

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Detection of More Than One Analyte Using a Same Probe

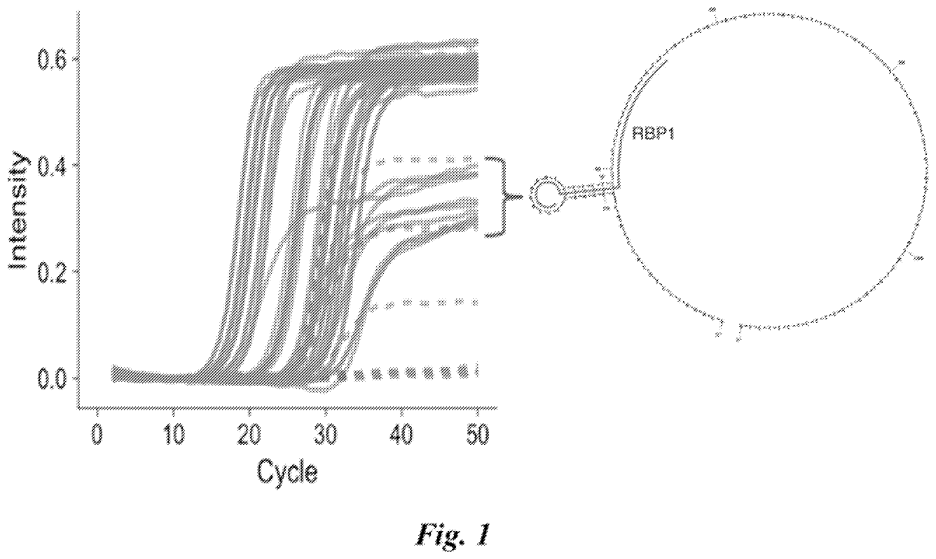

Samples comprising respiratory syncytial virus were analyzed for the presence of a variant and wild type strain. An oligonucleotide probe was added to the sample, along with reagents used for PCR. The oligonucleotide probe was able to hybridize to both strains of the RSV. The probe comprises a mismatch in a hairpin loop to the variant strain. A schematic of the probe and target are shown in FIG. 1, with a mismatch location indicated. The sample was subjected to a PCR reaction and the signal intensity was monitored. FIG. 1 shows curves on individual homogenous samples with some curves pertaining to a wild type strain and other curves pertaining to a variant strain. As observed in FIG. 1, two different groups of curves were generated with a first set of curves showing a fluorescence endpoint intensities of around 0.6 and a second set of curves with a fluorescence endpoint intensities of around 0.3. The wild type curves corresponded to the intensity of 0.6 whereas the variant curves corresponded to an intensity of 0.3, allowing for the differentiation of wild type strains from variant strains based on the curve shape and fluorescence endpoints and allowing the identification of the analytes present in each sample.

### Example 2: Detection of More Than One Analyte in a Digital PCR Reaction

A sample comprising a heterogeneous mixture of wild type sequences and a mutant sequence are added to a PCR mix comprising a probe that is able to hybridize to both wild type sequences and the mutant sequence. The sample is partitioned into multiple partitions and an amplification reaction is performed. A kinetic signature is generated for the partitions based at least on the intensity of signals generated during amplification cycles. Kinetic signatures of the partitions are compared to one another. Kinetic signature for partitions with mutant sequences demonstrate a different kinetic signature than partitions with wildtype sequences. Based on the differences in the kinetic signatures, the detection and quantification of the mutant sequences and wild type sequences is performed.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed. It is intended that the following claims define the scope of the invention and that methods and structures within the scope of these claims and their equivalents be covered thereby.

What is claimed is:

1. A method of determining the presence or absence of a first analyte and a second analyte in a sample or in a collection of samples, the method comprising:
a) providing a sample solution derived from a sample or collection of samples comprising, or potentially comprising, a first analyte and a second analyte;
b) adding an oligonucleotide configured to hybridize to said first analyte and said second analyte;
c) subjecting said sample solution to an extension reaction, wherein said extension reaction generates a first signal corresponding to said first analyte and/or a second signal corresponding to said second analyte;
d) detecting the presence or absence of said first signal and/or said second signal; and
e) based on said detecting, determining the presence or absence of said first analytes and/or said second analyte.

2. The method of claim 1, wherein said determining further comprises comparing said first signal and/or said second signal to a reference signal or a model of expected signals, and determining if said first signal and/or said second signal corresponds to said reference signal or model of expected signals.

3. The method of any one of claims 1-2, wherein said extension reaction comprises (i) hybridizing said oligonucleotide to said first analyte and/or said second analyte and (ii) extending said oligonucleotide.

4. The method of claim 3, wherein said first signal and/or said second signal is generated by the extension of said oligonucleotide.

5. The method of any one of claims 1-4, further comprising amplifying said first analyte or said second analyte under amplification conditions.

6. The method of claim 5, wherein said first signal corresponds with the amplification of said first analyte.

7. The method of claim 5 or 6, wherein said second signal corresponds with the amplification of said second analyte.

8. The method of any one of claims 5-7, wherein said amplifying comprises a polymerase chain reaction (PCR).

9. The method of claim 8, wherein said oligonucleotide acts as a primer in said PCR.

10. The method of any one of claims 1-9, wherein an intensity of a first signal corresponds to the efficiency of (i) hybridizing said oligonucleotide to said first analytes, and (ii) extension of said oligonucleotide.

11. The method of any one of claims 1-10, further comprising adding a plurality of probes to said sample solution.

12. The method of claim 11, wherein said plurality of probes generates said first signal or said second signal during said extension reaction.

13. The method of claim 11 or 12, wherein a probe of plurality of probes comprises a detectable label.

14. The method of any one of claims 11-13, wherein a probe of plurality of probes does not comprise a detectable label.

15. The method of claim 13 or 14, wherein said detectable label is a fluorophore.

16. The method of any one of claims 5-15, wherein said amplification conditions comprise one or more of thermocycling conditions, a salt concentration, a primer concentration, a deoxynucleotide triphosphate (dNTP) concentration, presence of a polymerase, and a template concentration.

17. The method of any one of claims 5-16, wherein under said amplification conditions, said amplifying of said first analyte occurs at a higher rate and/or efficiency compared to said amplifying of said second analyte.

18. The method of any one of claims 1-17, wherein said first signal, if generated has a higher intensity than said second signal, if generated.

19. The method of claim 18, wherein said determining said presence or absence of said first or second analyte comprises processing the intensity of said first signal or second signal.

20. The method of any one of claims 1-19, wherein said collection of samples is derived from a subject.

21. The method of any one of claims 1-20, wherein said collection of samples is derived from a plurality of subjects.

22. The method of any one of claims 1-21, wherein said subject is an biological organism or virus.

23. The method of claim 22, wherein the biological organism is a human, animal, plant, fungus, bacteria or archaea.

24. The method of claim 21, wherein (e) further comprises determining the presence or absence of said first analyte or second analyte in said plurality of subjects.

25. The method of any one of claims 20-24, wherein prior to (a), said collection of samples is pooled to generate said sample solution.

26. The method of any one of claims 1-25, wherein said second analyte is a variant of said first analyte.

27. The method of any one of claims 1-26, wherein said first analyte is a wild-type sequence and said second analyte is a mutant sequence.

28. The method of any one of claims 1-27, wherein said first and second analyte differ from one another by at least one nucleotide.

29. The method of any one of claims 1-28, wherein said first and second analyte differ from one another by at most one nucleotide.

30. The method of any one of claims 1-29, wherein said first and second analyte differ from one another by at a single nucleotide polymorphism.

31. The method of any one of claims 1-30, wherein said first and second analyte differ from one another by a plurality of single nucleotide polymorphisms.

32. The method of any one of claims 1-31, wherein said first and second analyte differ from one another by a deletion, insertion, transversion, duplication, or a combination thereof.

33. The method of claim 32, wherein said deletion is at least 1, 2, 3, 4, 5, 6, 7, 8, 9, 10, 15, 20, 25, or more nucleotides in length.

34. The method of claim 32, wherein said insertion is at least 1, 2, 3, 4, 5, 6, 7, 8, 9, 10, 15, 20, 25, or more nucleotides in length.

35. The method of claim 32, wherein said transversion is at least 1, 2, 3, 4, 5, 6, 7, 8, 9, 10, 15, 20, 25, or more nucleotides in length.

36. The method of claim 32, wherein said duplication is at least 1, 2, 3, 4, 5, 6, 7, 8, 9, 10, 15, 20, 25, or more nucleotides in length.

37. The method of any one of claims 1-36, wherein said first and second analytes comprise a sequence identity to one another of at least 80%, at least 90%, at least 95%, or at least 99%.

38. The method of any one of claims 1-37, wherein the method further comprises, prior to (c), partitioning the sample into a plurality of partitions.

39. The method of claim 37, wherein a partition of said plurality of partition comprises said first analyte or said second analyte.

40. The method of claim 37 or 39, wherein each partition of said plurality of partition comprises on average one copy of said first analyte and/or said second analyte.

41. The method of any one of claims 37-40, wherein each partition of said plurality of partition comprises one copy or less of said first analyte and/or said second analyte.

42. The method of any one of claims 37-41, wherein said partitioning comprises co-partitioning said oligonucleotide and said first analyte or said second analyte in a partition.

43. The method of any one of claims 39-42, wherein said first signal and/or said second signal comprises a kinetic signature for said partition.

44. The method of claim 43, further comprising comparing said kinetic signature for said partition with another kinetic signature of a partition of said subset of said partition.

45. The method of claim 44, wherein said comparing comprises determining the presence of said first analyte and said second analyte if said kinetic signature and said another kinetic signature differ.

46. The method of any one of claims 1-45, further comprising quantifying the amount first analyte or second analytes.

47. The method of any one of claims 1-46, further comprising quantifying a relative amount of first analyte to second analytes.

48. The method of any one of claims 1-47, wherein said first analyte or said second analyte corresponds to a disease condition in a source of said sample or collection of samples.

49. The method of any one of claims 1-47, wherein said sample or collection of samples is derived from wastewater.

50. The method of any one of claims 1-47, wherein said sample or collection of samples is derived from soil.

51. The method of any one of claims 1-50, wherein said first analyte or said second analyte corresponds to a genetic variant.

52. The method of any one of claims 1-51, wherein said first analyte or said second analyte comprises a human genomic sequence.

53. The method of any one of claims 1-52, wherein said first analyte or said second analyte comprises an animal genomic sequence.

54. The method of any one of claims 1-53, wherein said first analyte or said second analyte comprises a plant genomic sequence.

55. The method of any one of claims 1-54, wherein said first analyte or said second analyte comprises a fungal genomic sequence.

56. The method of any one of claims 1-55, wherein said first analyte or said second analyte comprises an archaeal genomic sequence.

57. The method of any one of claims 1-56, wherein said first analyte or said second analyte comprises a pathogen associated sequence.

58. The method of claim 57, wherein said pathogen-associated sequence comprises a viral sequence.

59. The method of claim 57 or 58, wherein said pathogen-associated sequence comprises a bacterial sequence.

60. The method of any one of claims 1-59, wherein said first analyte is derived from a first pathogen and the second analyte is derived from a second pathogen.

61. The method of claim 60, wherein said first pathogen and said second pathogen are a same pathogen.

62. The method of claim 60, wherein said first pathogen and said second pathogen are a different pathogen.

63. The method of any one of claims 60-62, wherein said first pathogen or said second pathogen is a coronavirus.

64. The method of claim 63, wherein said coronavirus is SARS-COV-2.

65. The method of any one of claims 60-64, wherein said first pathogen or said second pathogen is an influenza virus.

66. The method of any one of claims 60-65, wherein said first pathogen or said second pathogen is a respiratory syncytial virus.

67. The method of any one of claims 60-66, wherein said first pathogen or said second pathogen is a pathogen associated with respiratory infections.

68. The method of any one of claims 1-67, wherein said sample is derived from a plurality of cells.

69. The method of claim 68, wherein said plurality of cells are derived from a tumor.

70. The method of any one of claims 1-69, wherein said sample is a plasma sample or blood sample.

71. The method of any one of claims 1-70, wherein said sample comprises maternal nucleic acids and fetal nucleic acids

72. The method of any one of claims 1-71, wherein said first signal or second signal is an endpoint signal.

73. The method of any one of claims 1-72, further comprising, generating a kinetic signature from said first signal or said second signal.

74. The method of claim 73, wherein said kinetic signature comprises measuring a signal and a time point at a series of temperatures.

75. The method of claim 73 or 74, wherein said kinetic signature is represented as a curve based on endpoint signal over a series of temperatures.

76. The method of any one of claims 73-75, further comprising generating a kinetic signature from said first signal and said second signal

77. The method of any one of claims 1-76, wherein said oligonucleotide comprises a hairpin.

78. The method of any one of claims 1-77, wherein said oligonucleotide forms a hairpin upon hybridizing to said first analyte.

79. The method of any one of claims 1-78, wherein said oligonucleotide does not form a hairpin upon hybridizing to said second analyte.

80. The method of any one of claims 1-79, wherein said oligonucleotides is configured to form a hairpin in the presence of the first analyte, and not form a hairpin in the presence of said second analyte.

81. An oligonucleotide for determining the presence or absence of a first analyte and a second analyte, wherein
said oligonucleotide is configured to hybridize to said first analyte and said second analyte, wherein said oligonucleotide generates a first signal corresponding to said first analyte and a second signal corresponding to said second signal.

82. The oligonucleotide of claim 81, wherein said oligonucleotide is a primer.

83. The oligonucleotide of claim 82, wherein extension of said oligonucleotides generates said first signal and said second signal.

84. The oligonucleotide of claim 81, wherein said oligonucleotides is a probe.

85. The oligonucleotides of claim 84, wherein the oligonucleotide comprises a detectable label.

86. The oligonucleotide of any one of claims 81-85, wherein a hybridization efficiency of said oligonucleotide to said first analytes is different than a hybridization efficiency said oligonucleotide to said second analyte.

87. A kit for determining the presence or absence of a first analyte and a second analyte, wherein the kit comprises:
a. a primer oligonucleotide configured to hybridize to said first analyte and said second analyte, wherein said oligonucleotide generates a first signal corresponding to said first analyte and a second signal corresponding to said second analyte; and
b. a probe oligonucleotide configured to bind to said first analyte or said second analyte

88. A kit for determining the presence or absence of a first analyte and a second analyte, wherein the kit comprises:
a. a probe oligonucleotide configured to hybridize to said first analyte and said second analyte, wherein said probe oligonucleotide generates a first signal corresponding to said first analyte and a second signal corresponding to said second analyte; and
b. a set of primer oligonucleotide configured to bind to said first analyte or said second analyte.

89. A method of determining the presence or absence of a first analyte and a second analyte in a sample or in a collection of samples, the method comprising:
a) providing a sample solution comprising, or potentially comprising at a first analyte and a second analyte;
b) adding an oligonucleotide that is configured to hybridize to said first analyte and said second analyte;
c) subjecting said sample to an extension reaction, wherein said extension reaction generates: (i) a first signal if said first analyte is present or (ii) a second signal if said second analyte is present and the first analyte is not present;
d) detecting the first or second signal; and
e) based on said detecting of said first signal or said second signal, determining said presence or absence of said first and second analyte

90. A method of determining the presence or absence of a target nucleotide sequence, wherein the method comprises:
a) providing a sample solution comprising, or potentially comprising, an analyte sequence comprising said target nucleotide sequence and a second analyte comprising a reference sequence;
b) adding an oligonucleotide to a sample solution, wherein said oligonucleotide is configured to hybridize to a reference sequence and said target nucleotide sequence;
c) subjecting said sample to an extension reaction, wherein said extension reaction generates a signal corresponding to said target nucleotide sequence and a reference signal corresponding to said reference sequence;
d) detecting said signal and comparing said signal to a reference or model signal corresponding to said reference sequence; and
e) based on said comparing, determining the presence or absence of a target sequence.

91. A method of determining the presence or absence of a first analyte and a second analyte in a sample, the method comprising:
a) providing said sample;
b) adding to said sample an oligonucleotide configured to hybridize to said first analyte and said second analyte;
c) subjecting said sample to a first extension reaction at a first reaction condition, wherein said extension reaction generates a first plurality of signals corresponding to the extension products generated by said first extension reaction;
d) measuring, said first plurality of signals, if present;
e) subjecting said sample to a second extension reaction at a second reaction condition, wherein said extension reaction generates a second plurality of signals corresponding to the extension products generated;
f) measuring, said second plurality of signal, if present; and
g) based on the measuring of said first plurality of signals and said second plurality of signals, determining said presence or absence of said first and second analytes.

92. The method of claim 91, wherein said first reaction condition comprises a first annealing condition and said second reaction condition comprises second annealing condition.

93. The method of claim 92, wherein said first annealing condition is more stringent than said the second annealing condition.

94. The method of claim 92 or 93, wherein said first annealing condition comprises an annealing temperature that is greater than an annealing temperature of the second annealing condition.

95. The method of any one of claims 92-94, wherein said first annealing condition comprises an annealing time that is greater than an annealing time of the second annealing condition.

96. The method of any one of claims 91-95, wherein said first reaction condition comprises a first extension condition and said second reaction condition comprises second extension condition.

97. The method of claim 96, wherein said first extension condition or second extension condition is an extension time or temperature.

98. The method of any one of claims 92-97, wherein said first annealing condition comprises an annealing temperature that is greater than an annealing temperature of the second annealing condition.

99. The method of any one of claims 91-98, wherein in (c), said hybridization of said oligonucleotide to said first analyte is more favorable than hybridization of said oligonucleotide to said second analyte.

100. The method of any one of claims 91-99, wherein in (e), said oligonucleotide hybridizes to said first analyte and said second analyte.
